SEQUENCE LISTING (1 sequence)

SEQ ID NO: 1 - protein, 595 aa, Homo sapiens [sequence omitted]

(19) United States
(12) Patent Application Publication
(10) Pub. No.: US 2026/0315443 A1
(43) Pub. Date: Oct. 15, 2026

(54) IMLUNESTRANT OR SALTS THEREOF FOR USE IN TREATING AND PREVENTING CENTRAL NERVOUS SYSTEM (CNS) METASTASES IN SUBJECTS HAVING ER+ BREAST CANCER

(71) Applicant: ELI LILLY AND COMPANY, Indianapolis, IN (US)

(72) Inventors: Andrew Roy CAPEN, Greenfield, IN (US); Mark CASTANARES, Indianapolis, IN (US); Michele Suzanne DOWLESS, Pendleton, IN (US); Xueqian GONG, Carmel, IN (US); Lysiane HUBER, Danville, IN (US); Anke KLIPPEL-GIESE, Princeton, NJ (US); Loredana PUCA, New York, NY (US); Vivian RODRIGUEZ CRUZ, Clarksville, TN (US); Matthew James VANDE KOPPLE, Indianapolis, IN (US)

(21) Appl. No.: 19/481,050

(22) PCT Filed: May 2, 2024
(86) PCT No.: PCT/US2024/027427

§ 371 Date: Nov. 3, 2025

Related U.S. Application Data
(60) Provisional application No. 63/500,437, filed on May 5, 2023.

(51) Int. Cl. A61K 31/436; A61K 31/506; A61P 35/04

(52) U.S. Cl. CPC A61K 31/436; A61K 31/506; A61P 35/04

(57) ABSTRACT

The disclosure provides compositions and methods comprising imlunestrant of a pharmaceutically acceptable salt thereof for the treatment and prevention of brain metastasis. The disclosure provides, within such compositions and methods, for the treatment and/or the prevention of brain metastases in a subject suffering from ER+ breast cancer.

### BACKGROUND

Breast cancer is the most common cancer worldwide and the most common cancer diagnosed in American women. In 2020, about 2.3 million women were diagnosed with breast cancer worldwide and 685,000 died. Of these breast cancer cases, about 80% of breast cancers diagnosed are driven by the estrogen receptor (ER) which means that the cancer cells grow in response to the hormone estrogen (also known as ER+ breast cancer). As such, the estrogen receptor (ER) is a key therapeutic target for ER-positive (ER+) breast cancer. Novel degraders of ER may overcome resistance to available endocrine therapy (ET) while providing consistent oral bioavailability and convenience of administration. Imlunestrant (LY-3484356 or (R)-5-(4-(2-(3-(fluoromethyl)azetidin-1-yl)ethoxy)phenyl)-8-(trifluoromethyl)-5H-chromeno[4,3-c]quinolin-2-ol) is a novel, orally bioavailable selective estrogen receptor degrader (SERD) with pure antagonistic properties resulting in sustained inhibition of ER-dependent gene transcription and -cell growth. Imlunestrant is under clinical development and currently in a Phase III trial for metastatic breast cancer (NCT04188548, “A Study of LY3484356 in Participants With Advanced or Metastatic Breast Cancer or Endometrial Cancer (EMBER).”

In addition to being driven by ER, breast cancers also may be driven by the human epidermal growth factor receptor 2 (HER2) and may be characterized as HER2− negative (HER2−) or HER2-positive (HER2+). HER2− breast cancer represents the majority of breast cancers where only approximately 15-20% of breast cancers are characterized as HER2+. (Exman et al., Clin Adv Hematol Oncol. 2021 January; 19(1):40-50). However, HER2 status is dynamic and HER2− and HER2+ tumor cells may interconvert in response to treatment or spontaneously. (Jordan et al., Nature 537, 102-106 (2016).

Brain metastases (BM) may develop in up to 30-50% of patients with breast cancer and may have a significant impact on survival and quality of life. HER2 status is associated with an increased risk of metastases, and although brain metastases are observed in both ER+, HER2− and ER+, HER2+ metastatic breast cancer, patients with HER2+ breast cancer have an increased risk of developing brain metastases. In recent years, several novel anti-HER2 agents have led to significant improvements in the outcomes of HER2-positive metastatic breast cancer.

However, therapies for ER+ brain metastases that target the ER have limited effectiveness. These is at least partially because of the structure of the blood-brain barrier (BBB) between the cerebral capillary blood and the interstitial fluid of the brain. The BBB is made up of capillary endothelial cells and basement membrane, neuroglial membrane, and glial podocytes or projections of astrocytes. These three components work together to keep therapeutic compounds from reaching cancer cells that may have metastasized to the brain. Estrogen can cross the BBB which means that the growth of any ER+ cancer cells that have metastasized to the brain is not impacted by the lack of estrogen. A compound that could cross the BBB and antagonize or degrade the estrogen receptor such as a SERD would allow for the treatment and prevention of ER+ breast cancer metastases. A compound that could cross the BBB and antagonize or degrade the estrogen receptor such as a SERD also may reduce symptoms from metastasized ER+ breast cancers and may increase quality of life.

### SUMMARY

The disclosure relates to compounds, compositions and methods for preventing, reducing the occurrence and/or onset, of central nervous system (CNS) metastasis in a subject having cancer, and/or for inhibiting or preventing the survival of metastatic cancer cells in a subject having cancer, comprising administering to the subject an effective amount of a compound of Formula I, which may be referred to as imlunestrant, or a pharmaceutically acceptable salt, amorphous form, or polymorph form, thereof, or a composition comprising imlunestrant or a pharmaceutically acceptable salt, amorphous form, or polymorph form, thereof.

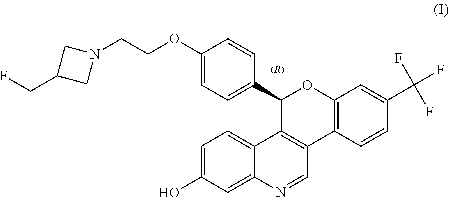

In an aspect, the disclosure relates to a method for preventing CNS metastasis in a subject having a cancer, comprising administering to the subject in need of prevention, imlunestrant or a pharmaceutically acceptable salt thereof, or a composition comprising imlunestrant or a pharmaceutically acceptable salt thereof in an amount effective to prevent CNS metastasis in the subject.

In another aspect, the disclosure relates to a method of reducing the risk of occurrence or development of CNS metastasis in a subject having a cancer, comprising administering to the subject in need thereof, imlunestrant or a pharmaceutically acceptable salt thereof, or a composition comprising imlunestrant or a pharmaceutically acceptable salt thereof in an amount effective to reduce the risk of occurrence of CNS metastasis in the subject.

In another aspect, the disclosure relates to a method of treating cancer that reduces the risk of developing detectable metastases of the cancer to the CNS, comprising administering to the subject having the cancer, imlunestrant or a pharmaceutically acceptable salt thereof, or a composition comprising imlunestrant or a pharmaceutically acceptable salt thereof in an amount effective to reduce the risk of detectable metastases in the CNS of the subject.

In another aspect, the disclosure relates to a method for delaying onset of CNS metastases in a subject having a cancer, comprising administering to the subject in need thereof, imlunestrant or a pharmaceutically acceptable salt thereof, or a composition comprising imlunestrant or a pharmaceutically acceptable salt thereof in an amount effective to delay onset of CNS metastases in the subject.

In a further aspect, the disclosure relates to a method of preventing onset of CNS metastases in a subject having a cancer, comprising administering to the subject in need thereof, imlunestrant or a pharmaceutically acceptable salt thereof, or a composition comprising imlunestrant or a pharmaceutically acceptable salt thereof in an amount effective to prevent onset of CNS metastases in the subject.

In a further aspect, the disclosure relates to a method for inhibiting metastatic progression of cancer in the CNS of a subject having a cancer, comprising administering to the subject in need thereof, imlunestrant or a pharmaceutically acceptable salt thereof, or a composition comprising imlunestrant or a pharmaceutically acceptable salt thereof in an amount effective to inhibit metastatic progression of cancer in the subject.

In another aspect, the disclosure relates to a method for inhibiting growth and/or survival of metastatic cancer cells in the CNS of a subject having a cancer, comprising administering to the subject in need thereof, imlunestrant or a pharmaceutically acceptable salt thereof, or a composition comprising imlunestrant or a pharmaceutically acceptable salt thereof in an amount effective to inhibit growth and/or survival of metastatic cancer cells in the CNS of the subject.

In another aspect, the disclosure relates to a method for inhibiting cancer from progressing to metastatic CNS cancer in a subject having a cancer, comprising administering to the subject in need thereof, imlunestrant or a pharmaceutically acceptable salt thereof, or a composition comprising imlunestrant or a pharmaceutically acceptable salt thereof in an amount effective to inhibit progression of the primary tumor to the CNS of the subject.

In another aspect, the disclosure relates to a method for reducing the risk of developing, or the onset of, detectable CNS metastases in a subject who has cancer, the method comprising administering to the subject in need thereof, imlunestrant or a pharmaceutically acceptable salt thereof, or a composition comprising imlunestrant or a pharmaceutically acceptable salt thereof in an amount effective to reduce the risk of developing, or the risk of onset of, detectable CNS tumors, such as, for example, CNS metastases in the subject.

In yet another aspect, the disclosure relates to a method for lengthening the period of survival of a subject having a cancer comprising administering to the subject in need thereof, imlunestrant or a pharmaceutically acceptable salt thereof, or a composition comprising imlunestrant or a pharmaceutically acceptable salt thereof in an amount effective to lengthen the period of survival of the subject, relative to the period of survival of the subject in the absence of administration.

In an aspect, the disclosure provides a compound of Formula (I) (i.e., imlunestrant) or a pharmaceutically acceptable salt, amorphous form, or polymorph form thereof, for use in the prevention of CNS tumors, such as, for example, in some particular embodiments for use in the prevention of CNS metastases, wherein the CNS metastases derive from a ER+ cancer.

In other aspects, the disclosure provides for the use of a compound of Formula (I) (i.e., imlunestrant) or a pharmaceutically acceptable salt, amorphous form, or polymorph form, thereof, or a composition comprising imlunestrant or a pharmaceutically acceptable salt, amorphous form, or polymorph form, thereof for reducing the risk of occurrence or development of CNS tumors, such as, for example, CNS metastases in a subject who has cancer.

In other aspects, the disclosure provides for the use of imlunestrant or a pharmaceutically acceptable salt, amorphous form, or polymorph form, thereof, or a composition comprising imlunestrant or a pharmaceutically acceptable salt, amorphous form, or polymorph form, thereof for reducing the risk of developing detectable CNS tumors, such as, for example, metastases of a cancer to the CNS in a subject who has cancer.

In further aspects, the disclosure provides for the use of imlunestrant or a pharmaceutically acceptable salt, amorphous form, or polymorph form, thereof, or a composition comprising imlunestrant or a pharmaceutically acceptable salt, amorphous form, or polymorph form, thereof for delaying onset of CNS tumors, such as, for example, CNS metastases in a subject who has cancer.

In yet other aspects, the disclosure provides for the use of imlunestrant or a pharmaceutically acceptable salt, amorphous form, or polymorph form, thereof, or a composition comprising imlunestrant or a pharmaceutically acceptable salt, amorphous form, or polymorph form, thereof for preventing onset of CNS tumors, such as, for example, CNS metastases in a subject who has cancer.

In other aspects, the disclosure provides for the use of imlunestrant or a pharmaceutically acceptable salt, amorphous form, or polymorph form, thereof, or a composition comprising imlunestrant or a pharmaceutically acceptable salt, amorphous form, or polymorph form, thereof for inhibiting CNS tumors, such as, for example, metastatic progression of cancer in the CNS of a subject who has cancer.

In other aspects, the disclosure provides for the use of imlunestrant or a pharmaceutically acceptable salt, amorphous form, or polymorph form, thereof, or a composition comprising imlunestrant or a pharmaceutically acceptable salt, amorphous form, or polymorph form, thereof for inhibiting growth and/or survival of CNS tumors, such as, for example, metastatic cancer cells in the CNS of a subject who has cancer.

In further aspects, the disclosure provides for the use of imlunestrant or a pharmaceutically acceptable salt, amorphous form, or polymorph form, thereof, or a composition comprising imlunestrant or a pharmaceutically acceptable salt, amorphous form, or polymorph form, thereof for inhibiting a cancer from progressing to CNS tumors, such as, for example, metastatic CNS cancer in a subject who has cancer.

In other aspects, the disclosure provides for the use of imlunestrant or a pharmaceutically acceptable salt, amorphous form, or polymorph form, thereof, or a composition comprising imlunestrant or a pharmaceutically acceptable salt, amorphous form, or polymorph form, thereof for reducing the risk of developing, or the onset of, CNS tumors, such as, for example, detectable CNS metastases in a subject who has cancer.

In another aspect, the disclosure provides for the use of compounds of Formula (I) (i.e., imlunestrant) or a pharmaceutically acceptable salt, amorphous form, or polymorph form, thereof, or a composition comprising imlunestrant or a pharmaceutically acceptable salt, amorphous form, or polymorph form, thereof for lengthening the period of survival of a subject who has cancer.

In any of the aspects relating to the use of compounds of Formula (I) (i.e., imlunestrant), the use may comprise the manufacture of a medicament, for preventing CNS tumors, such as, for example, CNS metastases in a subject who has cancer; for reducing the risk of occurrence or development of CNS tumors, such as, for example, brain metastases in a subject who has cancer; for reducing the risk of developing detectable CNS tumors, such as, for example, metastases of a cancer to the brain in a subject who has cancer; for delaying onset of CNS tumors, such as, for example, brain metastases in a subject who has cancer; for preventing onset of CNS tumors, such as, for example, brain metastases in a subject who has cancer; for inhibiting CNS tumors, such as, for example, metastatic progression of cancer in the brain of a subject who has cancer; for inhibiting growth and/or survival of CNS tumors, such as, for example, metastatic cancer cells in the brain of a subject who has cancer; for inhibiting a cancer from progressing to CNS tumors, such as, for example, metastatic brain cancer in a subject who has cancer; for reducing the risk of developing, or the onset of, detectable CNS tumors, such as, for example, brain metastases in a subject who has cancer; and/or for lengthening the period of survival of a subject who has cancer.

In an aspect, the disclosure provides the use of imlunestrant or a pharmaceutically acceptable salt, amorphous form, or polymorph form thereof, in the manufacture of a medicament. In embodiments of this aspect, the manufacture of a medicament may be for preventing CNS tumors, such as, for example, brain metastases, and in some particular embodiments for use in the manufacture of a medicament for the prevention of CNS tumors, such as, for example, brain metastases, wherein the CNS metastases derive from an ER+ cancer.

In one aspect, disclosed herein is imlunestrant or a pharmaceutically acceptable salt thereof, for use in the prevention of CNS metastases in a subject who has cancer.

In another aspect, disclosed herein is imlunestrant or a pharmaceutically acceptable salt thereof, for use in reducing the risk of occurrence or development of CNS metastases in a subject who has cancer.

In yet another aspect, disclosed herein is imlunestrant or a pharmaceutically acceptable salt thereof, for use in reducing the risk of developing detectable CNS metastases in a subject who has cancer.

In still yet another aspect, disclosed herein is imlunestrant or a pharmaceutically acceptable salt thereof, for use in delaying onset of CNS metastases in a subject who has cancer.

In another aspect, disclosed herein is imlunestrant or a pharmaceutically acceptable salt thereof, for use in preventing onset of CNS metastases in a subject who has cancer.

In still another aspect, disclosed herein is imlunestrant or a pharmaceutically acceptable salt thereof, for use in inhibiting metastatic progression of cancer in the CNS of a subject who has cancer.

In yet still another aspect, disclosed herein is imlunestrant or a pharmaceutically acceptable salt thereof, for use in inhibiting growth and/or survival of metastatic cancer cells in the CNS of a subject who has cancer.

In still yet another aspect, disclosed herein is imlunestrant or a pharmaceutically acceptable salt thereof, for use in inhibiting a cancer from progressing to metastatic CNS cancer in a subject who has cancer.

In yet another aspect, disclosed herein is imlunestrant or a pharmaceutically acceptable salt thereof, for use in reducing the risk of developing, or the onset of, detectable CNS metastases in a subject who has cancer.

In another aspect, disclosed herein is imlunestrant or a pharmaceutically acceptable salt thereof, for use in lengthening the period of survival of a subject who has cancer.

In the various aspects relating to imlunestrant or a pharmaceutically acceptable salt thereof for use: in preventing CNS tumors, such as, for example, brain metastases; in reducing the risk of occurrence or development of CNS tumors, such as, for example, brain metastases; in reducing the risk of developing detectable CNS tumors, such as, for example, metastases of a cancer to the brain in a subject who has cancer; in delaying onset of CNS tumors, such as, for example, brain metastases in a subject who has cancer; in preventing onset of CNS tumors, such as, for example, brain metastases in a subject who has cancer; in inhibiting CNS tumors, such as, for example, metastatic progression of cancer in the brain of a subject who has cancer; in inhibiting growth and/or survival of CNS tumors, such as, for example, metastatic cancer cells in the brain of a subject who has cancer; in inhibiting a cancer from progressing to CNS tumors, such as, for example, metastatic brain cancer in a subject who has cancer; in reducing the risk of developing, or the onset of, detectable CNS tumors, such as, for example, brain metastases in a subject who has cancer; and/or in lengthening the period of survival of a subject who has cancer, some embodiments relate to CNS tumors, such as, for example, brain metastases that derive from an ER+ cancer.

In some embodiments of any of the above aspects, the cancer comprises ER+ breast cancer, such as ER+, HER2− breast cancer.

In some embodiments of any of the above aspects, one or more cells of the cancer expresses an ER mutation. Expresses an ER protein having a deletion, insertion, substitution, relative to wild-type ER.

In some embodiments of any of the above aspects, the CNS tumors, such as, for example, brain metastases comprise an undetected or an undetectable metastasis.

In some embodiments of any of the above aspects, the subject has not had prior CNS tumors or metastases.

In some embodiments of any of the above aspects, the subject has had prior treatment for CNS tumors and/or prior metastases.

In some embodiments of any of the above aspects, the subject has advanced stage cancer.

In some embodiments of any of the above aspects, the subject has early-stage cancer.

In some embodiments of any of the above aspects, the subject has a primary cancer associated with an increased risk and/or an increased likelihood of metastatic progression to the CNS or more specifically, to the brain.

In some embodiments of any of the above aspects, the subject is not known to have CNS tumors, such as, for example, brain metastases prior to administering the methods.

In some embodiments of any of the above aspects, the method further comprises administering to the subject an additional anti-cancer agent.

In some embodiments of any of the above aspects, the subject is identified by detecting an ER mutation. Methods for detecting an ER mutation may include immunoassays or immunohistochemical assay, for example, whether the ER mutation is detected via detection of the expressed mutant ER (e.g., via an antibody that is specific for mutant ER relative to wild type). Methods for detecting an ER mutation may include nucleic acid assays, e.g., methods that include PCR amplification and sequencing to identify the ER mutation.

In some embodiments of any of the above aspects, the method or the use comprises once or twice daily administration of imlunestrant or a pharmaceutically acceptable salt, amorphous form, or polymorph form thereof, or a composition comprising imlunestrant or a pharmaceutically acceptable salt, amorphous form, or polymorph form, thereof.

In some embodiments of any of the above aspects, the method or use comprises imlunestrant or a pharmaceutically acceptable salt thereof in a polymorph form.

In some embodiments of any of the above aspects, the method or use comprises imlunestrant or a pharmaceutically acceptable salt thereof in an amorphous form.

In some embodiments of any of the above aspects, the method or use comprises imlunestrant as the free amine.

Other features, aspects, embodiments, and advantages of provided by the disclosure will be apparent from the detailed description that follows.

### BRIEF DESCRIPTION OF THE FIGURES

FIG. 1. ERα degradation assay by Western blot. (Top) Structure of imlunestrant. (Middle) Cells were treated for 72 hours with imlunestrant and total cell lysate subjected to Western blot for ERα. Blots were imaged using LI-COR Odyssey Classic Infrared Imaging System. Images were processed and analyzed using Image Studio version 3.1. (Bottom) Cells were treated with imlunestrant or fulvestrant with or without MG132, followed by Western blot analysis.

FIG. 2. Anti-proliferative activity of imlunestrant in a panel of ER+ and ER− breast cancer cell lines. A panel of ER+ and ER− breast cancer cell lines were treated for two doubling times with 10 serial dilutions of imlunestrant. Cells were fixed, then stained with propidium iodide (PI). Plates were scanned with an Acumen eX3 instrument and cell number was calculated with an Acumen algorithm. IC₅₀ values were determined by curve fitting the cell number data to a four-parameter logistic using GENE DATA™.

FIG. 3. Combination effect of: (a) imlunestrant and abemaciclib, (b) imlunestrant and alpelisib, and (c) imlunestrant and everolimus. Cell lines were treated with 10 serial dilutions of imlunestrant or indicated additional therapeutic agent alone or in combination. Cells were then fixed, stained with PI, and scanned with an Acumen eX3 instrument. IC₅₀ values from the single agent and combination treatment were used to calculate the combination index at 50% inhibition (CI₅₀).

FIG. 4. Combination cell viability and mechanism of action in MCF-7 cells. MCF7 cells were treated with imlunestrant, abemaciclib, or combination for 8 days. Cells were fixed, stained with primary antibody (KI67 or β-Gal), then a fluorescent secondary antibody and PI. TUNEL reagent was added to wells without antibody. Following wash, plates were read with an Acumen eX3 instrument. Proliferation data was based on cell counts and % positive data based on control population. (a) MCF-7 Proliferation; (b) MCF-7 Apoptosis; (c) MCF-7 Senescence; (d) MCF-7 Ki67.

FIG. 5. Drug exposure in plasma and various tissues. Mice bearing MCF7 xenografts were treated with imlunestrant for 3 days. Animals were sacrificed at the indicated time point, and organs and plasma collected for exposure analysis. (Top) 24 hrs post last dose, imlunestrant exposure demonstrated a dose dependent increase. (Bottom) RNA was prepared from half of each tumor to measure PGR inhibition by real-time qPCR analyses. GAPDH was used as the housekeeping gene to normalize results and PGR expression was calculated relative to the DMSO control for each sample.

FIG. 6. ERα and PgR immunohistochemical analysis. Tumors were collected, fixed with 10% neutral buffered formalin, and embedded in paraffin blocks for ERα and PGR immunohistochemical analysis. Blocks were then transferred to ARUP laboratories for processing, staining and analysis.

FIG. 7. In vivo efficacy in a CTG-2432 (ESR1 E380Q) xenograft model (Top) and a CTG-1211 (ESR1 D538G) xenograft model (Bottom). Mice were treated with vehicle, imlunestrant tosylate (15 mpk PO), abemaciclib (50 mpk PO), or fulvestrant (5 mpk) for 28 days. Imlunestrant and abemaciclib were dosed orally once per day, fulvestrant was dosed by subcutaneous injection once weekly (n=5 mice/group). Red line on graph represents treatment period.

FIG. 8. In vivo efficacy in: (a) a ST941/HI (ESR1 Y537S) xenograft model; (b) a ST941/PBR (ESR1 Y537S, palbociclib resistant) xenograft model; (c) a CTG-1260 (ESR1 D538G) xenograft model; (d) a T47D (ESR1 WT, PI3K H1047R) xenograft model; and (e) a ZR-75-1 (ESR1 WT, PI3K WT) xenograft model. Xenograft-bearing mice were treated with vehicle, imlunestrant tosylate or free base (5-15 mpk) and either alpelisib (30 mpk), abemaciclib (50 mpk), everolimus (5 mpk), or fulvestrant (5 mg/dose) and the combination of imlunestrant and additional therapeutic agents. All compounds were dosed orally once daily, except for fulvestrant which was dosed once weekly by subcutaneous injection (n=5 mice/group). Red line on graphs represents treatment period.

FIG. 9. Probability of survival, body weight, and total exposure in MCF brain orthotopic model. MCF-7 cells stably expressing luciferase (MCF7-luc) were implanted orthotopically into the brains of female NOD SCID mice supplemented with estrogen pellets. All compounds were dosed orally once daily, except for fulvestrant which was dosed once weekly by subcutaneous injection (n=10 mice/group). Red line on graphs represents treatment period. For exposure analysis, non-tumor bearing mice were dosed orally once daily for 7 days, except for fulvestrant, which was dosed once by SC injection. Brains from all treatment groups were harvested on day 8. (a) Probability of survival, (b) body weight, and (c) total exposure in MCF brain orthotopic model.

### DETAILED DESCRIPTION

CNS tumors, such as brain metastases, arise as a devastating progression of a variety of different cancers. Existing methods of prevention and treatment of CNS metastases are very limited. CNS metastases can occur when cancer cells spread from their original site (e.g., site of primary cancer) to the CNS such as the brain. Typically, CNS tumors, such as brain metastases, result when cancer cells travel through the bloodstream or the lymph system from the original tumor and spread (metastasize) to the CNS, such as the brain, and begin to proliferate.

Metastatic cancer that spreads from its original location is typically identified by the name of the primary cancer. For example, cancer that has spread from the breast to the brain is called metastatic breast cancer, not brain cancer. While any cancer can spread to the brain, some cancers have an increased likelihood of causing brain metastases, including the non-limiting example of breast cancer. CNS tumors, such as brain metastases, occur in 10 to 30 percent of adults with cancer. As the metastatic CNS tumors, e.g., brain tumors grow, they create pressure on and change the function of surrounding brain. As such, CNS tumors and brain metastases can be identified by a number of signs and symptoms. For example, signs and symptoms of brain metastases can include, headache, including headache with vomiting or nausea, cognitive changes including, for example increases in occurrence of memory problems, seizure, dizziness, and the like.

As described in more detail below, the disclosure is based on surprising results demonstrating that imlunestrant can cross the blood brain barrier and may be administered to treat established brain cancer and brain metastases. Because imlunestrant can cross the blood brain barrier, imlunestrant also may be effective in preventing brain metastases in subjects who are at risk of developing brain metastases.

### Definitions

Unless defined otherwise, all technical and scientific terms used herein have the meaning commonly understood by a person skilled in the art to which this invention belongs. As used herein, the following terms have the meanings ascribed to them below, unless specified otherwise.

CNS tumors are tumors of the central nervous system and include, but are not necessarily limited to brain or spinal cord tumors and brain or spinal cord metastases.

The term “polymorph,” as used herein, refers to crystals of the same compound having different physical properties as a result of the order of the molecules in the crystal lattice. Different polymorphs of a single compound have one or more different chemical, physical, mechanical, electrical, thermodynamic, and/or biological properties from each other. Differences in physical properties exhibited by polymorphs can affect pharmaceutical parameters such as storage stability, compressibility, density (important in composition and product manufacturing), dissolution rates (an important factor in determining bio-availability), solubility, melting point, chemical stability, physical stability, powder flowability, water sorption, compaction, and particle morphology. Differences in stability can result from changes in chemical reactivity (e.g., differential oxidation, such that a dosage form discolors more rapidly when comprised of one polymorph than when comprised of another polymorph) or mechanical changes (e.g., crystal changes on storage as a kinetically favored polymorph converts to a thermodynamically more stable polymorph) or both (e.g., one polymorph is more hygroscopic than the other). As a result of solubility/dissolution differences, some transitions affect potency and/or toxicity. In addition, the physical properties of the crystal may be important in processing; for example, one polymorph might be more likely to form solvates or might be difficult to filter and wash free of impurities (i.e., particle shape and size distribution might be different between one polymorph relative to the other). “Polymorph”, as used herein, does not include amorphous forms of the compound. As used herein, “amorphous” refers to a noncrystalline form of a compound which can be a solid state form of the compound or a solubilized form of the compound. For example, “amorphous” refers to a compound (e.g., a solid form of the compound) without a regularly repeating arrangement of molecules or external face planes.

The term “anhydrous,” as used herein, refers to a crystal form of the compound of Formula (I) that has 1% or less by weight water. For example, 0.5% or less, 0.25% or less, or 0.1% or less by weight water.

The term “solvate” as used herein refers to a crystalline form of the compound of Formula (I), such as a polymorph form of the compound, where the crystal lattice comprises one or more solvents of crystallization.

The terms “hydrate” or “hydrated polymorph form” refer to a crystalline form of the compound of Formula (I), such as a polymorph form of the compound, where the crystal lattice comprises water. Unless specified otherwise, the term “hydrate” as used herein refers to a “stoichiometric hydrate.” A stoichiometric hydrate contains the water molecules as an integral part of the crystal lattice, where removal of the water molecules will cause instability of the crystal network. In comparison, a non-stoichiometric hydrate comprises water, but changes in the water content does not cause significant changes to the crystal structure. During drying of non-stoichiometric hydrates, a considerable proportion of water can be removed without significantly disturbing the crystal network, and the crystals can subsequently rehydrate to give the initial non-stoichiometric hydrated crystalline form. Unlike stoichiometric hydrates, the dehydration and rehydration of non-stoichiometric hydrates is not accompanied by a phase transition, and thus all hydration states of a non-stoichiometric hydrate represent the same crystal form.

“Purity,” when used in reference to a composition including a polymorph of the compound of Formula (I), refers to the percentage of one specific polymorph form relative to another polymorph form or an amorphous form of the compound of Formula (I) in the referenced composition. For example, a composition comprising polymorph Form 1 having a purity of 90% would comprise 90 weight parts Form 1 and 10 weight parts of other polymorph and/or amorphous forms of the compound of Formula (I).

As used herein, a compound or composition is “substantially free of” one or more other components if the compound or composition contains no significant amount of such other components. For example, the composition can contain less than 5%, 4%, 3%, 2%, or 1% by weight of other components. Such components can include starting materials, residual solvents, or any other impurities that can result from the preparation of and/or isolation of the compounds and compositions provided herein. In some embodiments, a polymorph form provided herein is substantially free of other polymorph forms. In some embodiments, a particular polymorph of the compound of Formula (I) is “substantially free” of other polymorphs if the particular polymorph constitutes at least about 95% by weight of the compound of Formula (I) present. In some embodiments, a particular polymorph of the compound of Formula (I) is “substantially free” of other polymorphs if the particular polymorph constitutes at least about 97%, about 98%, about 99%, or about 99.5% by weight of the compound of Formula (I) present. In certain embodiments, a particular polymorph of the compound of Formula (I) is “substantially free” of water if the amount of water constitutes no more than about 2%, about 1%, or about 0.5% by weight of the polymorph.

As used herein, “substantially pure,” when used in reference to a polymorph form of the compound of Formula (I), means a sample of a polymorph form of the compound having a purity greater than 90%, including greater than 90%, 91%, 92%, 93%, 94%, 95%, 96%, 97%, 98%, and 99%, and also including equal to about 100% of the compound, based on the weight of the compound. The remaining material comprises other form(s) of the compound, and/or reaction impurities and/or processing impurities arising from its preparation. For example, a polymorph form of the compound of Formula (I) may be deemed substantially pure in that it has a purity greater than 90% of a polymorph form of the compound of Formula (I), as measured by means that are at this time known and generally accepted in the art, where the remaining less than 10% of material comprises other form(s) of the compound of Formula (I) and/or reaction impurities and/or processing impurities. The presence of reaction impurities and/or processing impurities may be determined by analytical techniques known in the art, such as, for example, chromatography, nuclear magnetic resonance spectroscopy, mass spectrometry, or infrared spectroscopy.

To provide a more concise description, some of the quantitative expressions herein are recited as a range from about amount X to about amount Y. It is understood that when a range is recited, the range is not limited to the recited upper and lower bounds, but rather includes the full range from about amount X through about amount Y, or any range therein.

“Room temperature” or “RT” refers to the ambient temperature of a typical laboratory, which is typically around 25° C.

As used herein, the term “excipient” refers to any substance needed to formulate the composition to a desired form. For example, suitable excipients include but are not limited to, diluents or fillers, binders or granulating agents or adhesives, disintegrants, lubricants, antiadherants, glidants, dispersing or wetting agents, dissolution retardants or enhancers, adsorbents, buffers, chelating agents, preservatives, colors, flavors and sweeteners.

The term “pharmaceutically acceptable carrier” or “pharmaceutically acceptable excipient” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is well known in the art. Except insofar as any conventional media or agent is incompatible with the active ingredient, its use in the therapeutic formulations is contemplated. Supplementary active ingredients can also be incorporated into the formulations. In addition, various excipients, such as are commonly used in the art, can be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

As used herein, the singular forms “a,” “an,” and “the” include plural referents unless the context clearly dictates otherwise.

As used herein, ranges and amounts can be expressed as “about” a particular value or range. About also includes the exact amount. Hence, “about 5 grams” means “about 5 grams” and also “5 grams.” It also is understood that ranges expressed herein include whole numbers within the ranges and fractions thereof. For example, a range of between 5 grams and 20 grams includes whole number values such as 5, 6, 7, 8, 9, 10, 11, 12, 13, 14, 15, 16, 17, 18, 19 and 20 grams, and fractions within the range including, but not limited to, 5.25, 6.5, 8.75 and 11.95 grams. The term “about” preceding a value, which are reported as degrees Celsius, have an allowable variability of +1-5° C.

As used herein, “optional” or “optionally” means that the subsequently described event or circumstance does or does not occur and that the description includes instances where said event or circumstance occurs and instances where it does not. For example, a reaction mixture that “optionally includes a catalyst” means that the reaction mixture contains a catalyst or it does not contain a catalyst.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

All combinations of the embodiments pertaining to the aspects described herein are specifically embraced by the present invention just as if each and every combination was individually explicitly recited, to the extent that such combinations embrace possible aspects. In addition, all sub-combinations of the embodiments contained within the aspects described herein, as well as all sub-combinations of the embodiments contained within all other aspects described herein, are also specifically embraced by the present invention just as if each and every sub-combination of all embodiments are explicitly recited herein.

### Methods & Uses

Compounds of Formula (I) or a pharmaceutically acceptable salt, amorphous form, or polymorph form thereof, or pharmaceutical composition (e.g., any of the solid or liquid formulations described herein) thereof, can act as selective estrogen receptor degraders (SERDs) as demonstrated by the assays described in, e.g., PCT Publication No. WO2020/014435, which is incorporated by reference in its entirety.

As described throughout the disclosure, a compound of Formula I (imlunestrant), as provided herein, can exhibit brain and/or central nervous system (CNS) penetrance. In some embodiments of the methods disclosed herein, the compounds are capable of crossing the blood brain barrier and acting as a SERD in the brain and/or other CNS structures. In some embodiments, the compounds provided herein are capable of crossing the blood brain barrier in an amount effective to prevent a disease. In some embodiments, the compounds provided herein are capable of crossing the blood brain barrier in a therapeutically effective amount. For example, treatment of a patient with cancer (e.g., a cancer having metastatic potential, a cancer having a risk of metastatic spread, a cancer likely to develop metastases in the brain or CNS) can include administration (e.g., oral administration) of the compound to the patient. In some such embodiments, the compounds provided herein are useful for treating a metastatic brain tumor. For example, a breast cancer that has metastasized to the brain of a subject.

Various aspects and embodiments of the disclosure relate to methods for preventing a brain metastasis. “Metastasis” or the plural, “metastases”, as used herein, refers to the presence of one or more cancer cells at a location that is not physically contiguous with the original location of the cancer (e.g., primary cancer). For example, and as discussed in greater detail below, the primary cancer can include, but is not limited to, breast cancer.

A metastatic growth, lesion, or cluster of cells may be detectable using any known method for providing an identification or diagnosis of metastases, including, for example magnetic resonance imaging, computerized tomography or positron emission tomography. In some embodiments, a cluster of metastatic cells may include at least about 1×10⁷ cells. In some embodiments, a detectable metastasis can include a cluster of cells having a size greater than about 5 mm or about 10 mm. In some embodiments, the disclosure provides methods for preventing brain metastases in a subject who is at risk of developing brain metastases and who has no detectable metastatic growth and/or no diagnosis of metastatic growth. Thus, in some embodiments, the methods of preventing brain metastases comprise administering a compound of Formula I to a subject who has a risk and/or increase likelihood or chance of developing brain metastases.

Thus, in embodiments of the methods disclosed herein, compounds of Formula (I) (i.e., imlunestrant) or a pharmaceutically acceptable salt, amorphous form, polymorph form, or pharmaceutical composition (e.g., any of the solid or liquid formulations described herein) thereof are useful for preventing brain metastases. The term “preventing” as used herein means the prevention or delaying of the onset, recurrence or spread, in whole or in part, of the disease or condition as described herein, e.g., brain metastases, or a symptom thereof. In some embodiments, “preventing” can also mean prolonging survival as compared to expected survival if not receiving the preventative method/therapy.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As described in detail herein, including the illustrative Examples below, the disclosure demonstrates that methods comprising administering a compound of Formula (I) or a pharmaceutically acceptable salt, amorphous form, polymorph form, or pharmaceutical composition (e.g., any of the solid or liquid formulations described herein) thereof to a patient in need thereof can be used to prevent and/or treat brain metastasis. Accordingly, in various aspect and embodiments, the disclosure provides compounds, compositions, and dosage forms comprising a compound of Formula (I) (imlunestrant) or a pharmaceutically acceptable salt, amorphous form, polymorph form, or pharmaceutical composition (e.g., any of the solid or liquid formulations described herein), and methods that comprise administration of the compound/pharmaceutical composition that are effective to inhibit the onset and the progression of brain metastatic lesions.

In some embodiments, the disclosure provides a method of treating a subject having a cancer comprising administering an amount of a compound of Formula (I) (i.e., imlunestrant) or a pharmaceutically acceptable salt, amorphous form, polymorph form, or pharmaceutical composition (e.g., any of the solid or liquid formulations described herein) thereof, to a subject in need thereof in an amount that inhibits metastatic progression of the cancer to and/or in the brain. In some embodiments, the cancer can comprise breast cancer. In some example embodiments, one or more cells of the cancer of the subject expresses an ESR1 mutation. In some embodiments, the subject may receive a diagnosis that identifies the cancer as ER+ cancer. In some embodiments, the subject may receive a diagnosis that identifies the cancer as HER2+ cancer. In some embodiments, the subject may receive a diagnosis that identifies the cancer as HER2− cancer. In some embodiments, the subject may receive a diagnosis or may be identified as having an increased risk of developing brain metastases prior to receiving the preventative method. In some embodiments, the subject is not known to have a brain metastasis prior to treatment. In further embodiments, the imlunestrant is an imlunestrant salt, such as imlunestrant tosylate. In further embodiments, the imlunestrant is the free amine, i.e., not a pharmaceutically acceptable salt.

In embodiments, the disclosure provides a method for treating a subject having a cancer comprising administering, to the subject, an amount of a compound of Formula (I) (i.e., imlunestrant) or a pharmaceutically acceptable salt, amorphous form, polymorph form, or pharmaceutical composition (e.g., any of the solid or liquid formulations described herein) thereof, effective to inhibit metastatic progression of the cancer in the brain.

In some embodiments, the disclosure provides a method for preventing brain metastases in a subject having a cancer comprising administering, to the subject, an amount of a compound of Formula (I) (i.e., imlunestrant) or a pharmaceutically acceptable salt, amorphous form, polymorph form, or pharmaceutical composition (e.g., any of the solid or liquid formulations described herein) thereof, effective to inhibit metastatic progression of the cancer in the brain.

In some embodiments, the disclosure provides a method for inhibiting growth and/or survival of metastatic cancer cells in the brain of a subject, comprising administering, to the subject, an effective amount of a compound of Formula (I) (i.e., imlunestrant) or a pharmaceutically acceptable salt, amorphous form, polymorph form, or pharmaceutical composition (e.g., any of the solid or liquid formulations described herein) thereof. In certain embodiments, the cancer comprises breast cancer, e.g., ER+ breast cancer. In some further embodiments, one or more cells of the cancer of the subject express an ESR1 mutation that is associated with a cancer.

In some embodiments, the disclosure provides a method of treating brain metastases in a subject comprising administering, to the subject, a therapeutically effective amount of a compound of Formula (I) (i.e., imlunestrant) or a pharmaceutically acceptable salt, amorphous form, polymorph form, or pharmaceutical composition (e.g., any of the solid or liquid formulations described herein) thereof. In some embodiments, the brain metastases cancer is associated with a primary cancer that comprises breast cancer.

In further embodiments, one or more cells of the cancer of the subject express an ESR1 mutation that is associated with the primary cancer and/or the brain metastases. In further embodiments, the brain metastases may comprise a detectable metastasis. In some other further embodiments, the brain metastasis may comprise an undetected or an undetectable metastasis.

In some embodiments, the disclosure provides for a method of preventing metastasis of a cancer to the brain in a subject in need of prevention, comprising administering, to the subject, a therapeutically effective amount of a compound of Formula (I) (i.e., imlunestrant) or a pharmaceutically acceptable salt, amorphous form, polymorph form, or pharmaceutical composition (e.g., any of the solid or liquid formulations described herein) thereof. In certain embodiments, the cancer comprises breast cancer such as ER+ breast cancer. In further embodiments, one or more cells of the cancer of the subject express an ESR1 mutation that is associated with a cancer.

In some embodiments, the disclosure provides for a method of reducing the risk of developing, or the onset of, detectable brain metastasis in a subject who has cancer, the method comprising administering, to the subject in need of reducing risk, an effective amount of a compound of Formula (I) (i.e., imlunestrant) or a pharmaceutically acceptable salt, amorphous form, polymorph form, or pharmaceutical composition (e.g., any of the solid or liquid formulations described herein) thereof. In some embodiments, the cancer comprises breast cancer such as ER+ breast cancer. In further embodiments, one or more cells of the cancer of the subject express an ESR1 mutation that is associated with a cancer.

In some embodiments, the disclosure provides a method for lengthening the period of survival of a subject having a cancer comprising administering, to the subject, an effective amount of a compound of Formula (I) (i.e., imlunestrant) or a pharmaceutically acceptable salt, amorphous form, polymorph form, or pharmaceutical composition (e.g., any of the solid or liquid formulations described herein) thereof. In some embodiments, the cancer comprises breast cancer such as ER+ breast cancer. In further embodiments, one or more cells of the cancer of the subject express an ESR1 mutation that is associated with a cancer. In some embodiments, the subject has an advanced stage of disease (i.e., cancer), such as an advanced form of breast cancer such as ER+ breast cancer. In some embodiments, the subject having an advanced stage of cancer was known to have one or more brain metastases prior to treatment.

In some embodiments, the subject does not have advanced stage breast cancer. In some embodiments, the subject may have early-stage breast cancer.

In some embodiments, the methods described herein can lengthen the survival period of a subject having cancer by, for example, a period of months to years (e.g., about 1 month, about 2 months, about 3 months, about 4 months, about 6 months, about 8 months, about 10 months, about 12 months, about 14 months, about 18 months, about 20 months, about 2 years, about 3 years, about 4 years, about 5 years, about 6 years or more).

In some embodiments of the methods described above, the subject may be known to have one or more metastases (e.g., brain or other mestastases) prior to treatment, or may have been previously treated for prior metastases (i.e., brain or other metastases). In some embodiments of the methods described above, the subject is not known to have a brain metastasis prior to the administering a treatment or preventive method.

In some embodiments, the disclosure provides for a method for treating cancer cell metastasis in a subject in need of such treatment, comprising administering, to the subject, an effective amount of a compound of Formula (I) (i.e., imlunestrant) or a pharmaceutically acceptable salt, amorphous form, polymorph form, or pharmaceutical composition (e.g., any of the solid or liquid formulations described herein) thereof.

In some embodiments, the disclosure provides a method of producing an anti-cancer effect in a subject having a cancer comprising administering, to the subject, an effective amount of a compound of Formula (I) (i.e., imlunestrant) or a pharmaceutically acceptable salt, amorphous form, polymorph form, or pharmaceutical composition (e.g., any of the solid or liquid formulations described herein) thereof.

In various embodiments of the methods described above, the disclosure provides: for treating a subject who has cancer, for inhibiting the growth and/or survival of cancer cells, for preventing and/or delaying the reoccurrence of a cancer, for inhibiting the infiltration of cancer cells and for lengthening the period of survival of a subject having cancer, comprising, administering, to the subject, a therapeutically effective amount of a compound of Formula (I) (i.e., imlunestrant) or a pharmaceutically acceptable salt, amorphous form, polymorph form, or pharmaceutical composition (e.g., any of the solid or liquid formulations described herein) thereof.

Also provided herein is a compound of Formula (I) or a pharmaceutically acceptable salt, amorphous form, polymorph form, for use in the various indications discussed above (e.g., prevention of ER+ brain metastases). Brain metastases that can be prevented in accordance with the aspects and embodiments comprising the use of the compound of Formula (I), or a pharmaceutically acceptable salt, amorphous form, polymorph form are described herein. In some embodiments, the use of the compound of Formula (I), or a pharmaceutically acceptable salt, amorphous form, polymorph form relating to prevention of ER+ associated brain metastases can also comprise performing an in vitro assay using a biological sample from a patient, determining or detecting the presence of wild-type ER or mutant ER (i.e., a cancer having one or more mutations in ESR1), and administering a prophylactically effective amount of the compound of Formula (I) or a pharmaceutically acceptable salt, amorphous form, polymorph form thereof, to the patient if the wild-type ER or mutant ER is determined or detected. In such uses, the biological sample can be a tumor sample and the tumor sample can be analyzed using methods known to those of skill in the art such as genomic/DNA sequencing. Additionally, in such uses the sample can be obtained from the patient prior to the first administration of the compound of Formula (I) or a pharmaceutically acceptable salt, amorphous form, polymorph form thereof. In these uses of the compound of Formula (I) or a pharmaceutically acceptable salt, amorphous form, polymorph form, the use/administration can be based upon a patient being selected by having wild-type ER expression or mutant ER expression. In other embodiments of these uses, the use/administration can be based upon a patient being suspected of having, including suspected of having an increased likelihood of having, a cancer expressing wild-type ER or mutant ER. In any embodiments of these uses a compound of Formula (I) or a pharmaceutically acceptable salt, amorphous form, polymorph form, as described herein, may be administered to the patient at a dose of about 0.1 mg/kg to 200 mg/kg (effective dosage sub-ranges are noted herein).

In some embodiments, the methods and uses described herein can further comprise administering to the subject an additional anti-cancer agent, as is generally known in the art. In some example embodiments, the methods described herein, may comprise administering, to the subject, a therapeutically effective amount of imlunestrant or a pharmaceutically acceptable salt, amorphous form, polymorph form, or pharmaceutical composition (e.g., any of the solid or liquid formulations described herein) thereof, and an effective amount of an anti-cancer agent that can penetrate the blood brain barrier to achieve therapeutic levels, such as (for example), nimustine (N′-[(4-amino-2-methylpyrimidin-5-yl)methyl]-N-(2-chloroethyl)-N-nitrosourea or ACNU), carmustine (bis-chloroethylnitrosourea, BiCNU, or BCNU), lomustine (1-(2-chloroethyl)-3-cyclohexyl-1-nitrosourea or CCNU), hydroxyurea, topotecan, temozolomide, dacarbazine, methotrexate, cytarabine (cytosine arabinoside or ara-C), capecitabine, cisplatin, vinorelbine, carboplatin, or combinations thereof.

In some embodiments of the disclosed methods, imlunestrant is administered to a subject having brain metastasized breast cancer (BMBC) and symptoms of BMBC are treated or prevented. Symptoms of BMBC may include headaches, memory loss, changes in mood, changes in behavior, changes in personality, impaired judgment, dizziness, balance problems, weakness or paralysis in one side of the body (hemiparesis), numbness or tingling within the extremities, blurred vision, double vision, loss of vision, seeing flashes of light, slurred speech, nausea, vomiting, strokes, and seizures. The disclosed methods may reduce the severity of the symptoms or stop the symptoms entirely. Symptoms of BMBC may be assessed using methods as known in the art including neurological tests, balance and strength tests, and vision tests.

In some embodiments of the disclosed methods, imlunestrant is administered to a subject having brain metastasized breast cancer (BMBC) characterized by one or more brain tumors. The disclosed methods may reduce the size of the one or more brain tumors. In some embodiments the disclosed methods, a subject having a brain tumor is administered imlunestrant for at least about 1, 2, 3, 4, 5, 6, 7, or 8 weeks. In some embodiments of the disclosed methods, after the subject having a brain tumor has been administered imlunestrant for at least about 1, 2, 3, 4, 5, 6, 7, or 8 weeks, the size of the brain tumor is reduced by at least 10%, 20%, 30%, 40%, 50%, 60%, 70%, 80%, 90%, or more to the point where the brain tumor is no longer detectable. Measurement of the size of brain tumor and reduction in the size of brain tumor may be assessed using methods as known in the art which may include magnetic resonance imaging (MRI) and positron emission tomography (PET).

In some embodiments of the disclosed methods, imlunestrant is administered to a subject who has ER+ breast cancer and who may have brain metastasized ER+ breast cancer and the subject's overall survival time (OS) is extended, for example, in comparison to a subject who was not administered imlunestrant or who was administered a SERD that is not imlunestrant. For example, the disclosed methods may extend overall survival of the subject of the methods. Overall survival (OS) may be defined as the time from first administration of imlunestrant to the subject until death.

In some embodiments of the disclosed methods, imlunestrant is administered to a subject who has ER+ breast cancer and who may have brain metastasized ER+ breast cancer and the subject's progression-free survival (PFS) time is extended, for example, in comparison to a subject who was not administered imlunestrant or who was administered a SERD that is not imlunestrant. For example, the disclosed methods may extend progression-free survival of the subject of the methods. Progression free survival (PFS) may be defined as the time from first administration of imlunestrant to the subject until first evidence of disease progression or death. Disease progression may include tumor growth or spread in the brain.

In some embodiments of the disclosed methods, imlunestrant is administered to a subject who has ER+ breast cancer and who may have brain metastasized ER+ breast cancer and the subject's time to progression (TPP) is extended, for example, in comparison to a subject who was not administered imlunestrant or who was administered a SERD that is not imlunestrant. Time to progression (TTP) may be defined as the time from first administration of imlunestrant to the subject until first evidence of disease progression. Disease progression may include tumor growth or spread in the brain.

In some embodiments of the disclosed methods, imlunestrant is administered to a subject who has ER+ breast cancer and who may have brain metastasized ER+ breast cancer and the subject's disease free survival time (DFS) is extended, for example, in comparison to a subject who was not administered imlunestrant or who was administered a SERD that is not imlunestrant. Disease free survival time (DFS) may be defined as the time from first administration of imlunestrant to the subject until first evidence of disease recurrence. Disease recurrence may include recurrence of brain metastases.

In some embodiments of the disclosed methods, imlunestrant is administered to a subject who has ER+ breast cancer and who may have brain metastasized ER+ breast cancer and the subject's event-free survival time (EFS) is extended, for example, in comparison to a subject who was not administered imlunestrant or who was administered a SERD that is not imlunestrant. Event-free survival (EFS) may be defined as the time from first administration of imlunestrant to the subject to an event which may include disease progression, discontinuation of the treatment for any reason, or death. Disease progression may include include tumor growth or spread in the brain.

In some embodiments of the disclosed methods, imlunestrant is administered to a subject who has ER+ breast cancer and who may have brain metastasized ER+ breast cancer and the subject may achieve an improvement in health-related quality of life (HRQoL), for example, in comparison to a subject who was not administered imlunestrant or who was administered a SERD that is not imlunestrant. Health-related quality of life (HRQoL) is patient reported and may demonstrate clinical benefit. HRQoL may be assessed using a set of four core questions developed by the Centers for Disease Control and Prevention (CDC), which include concepts related to overall health, physical health, mental health, and activities of daily living.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of a disease or disorder to be treated and/or prevented. In some embodiments, the subject has been identified or diagnosed as having a cancer, such as ER+ breast cancer (e.g., as determined using a regulatory agency-approved, e.g., an FDA-approved, assay or kit or a lab developed test (LDT)). In some embodiments, the subject has a tumor that is positive for wild-type ER or mutant ER expression or activity (e.g., as determined using a regulatory agency-approved assay or kit). The subject can be a subject with a tumor(s) that is positive for wild-type ER or mutant ER expression or activity (e.g., identified as positive using a regulatory agency-approved, e.g., FDA-approved, assay or kit). The subject can be a subject whose tumors have a dysregulation of wild-type ER or mutant ER expression or activity (e.g., where the tumor is identified as such using a regulatory agency-approved, e.g., FDA-approved, kit or assay). In some embodiments, the subject is suspected of having a wild-type ER or mutant ER associated cancer. In some embodiments, the subject has a clinical record indicating that the subject has a tumor that has wild-type ER or mutant ER expression or activity (and optionally the clinical record indicates that the subject should be treated with any of the compositions provided herein).

In some embodiments of any of the methods or uses described herein, the primary cancer (e.g., an ER+ cancer) is a solid tumor. In some embodiments of any of the methods or uses described herein, the primary cancer (e.g., an ER+ cancer) is breast cancer, mammary cancer, mammary carcinoma, mammary neoplasm, or pregnancy-associated breast cancer.

In embodiments relating to methods for preventing brain metastases as described herein, a subject may be identified as having a risk, including an increased risk, of developing or onset one or more metastatic growths in the brain. In some embodiments, a subject may be identified by establishing and/or determining a stage of underlying disease, such as identifying stage of primary (or underlying) cancer disease. In some embodiments, a subject may be identified by having an underlying disease, e.g., a type of cancer, with a known risk of progressing to metastatic growth. In some embodiments, a subject may be identified by detecting and/or determining that the primary (or underlying) cancer disease is an ER+ associated cancer.

In some embodiments of the disclosed methods, the imlunestrant that is administered to a subject in need thereof binds to wild-type or mutant ER. In some embodiments of the disclosed methods, the imlunestrant that is administered to a subject in need thereof exhibits potent and selective inhibition of wild-type or mutant ER, e.g., by inducing degradation of the wild-type or mutant ER as a selective estrogen receptor degrader (SERD). In some embodiments of the disclosed methods, the imlunestrant that is administered to a subject in need thereof may exhibit nanomolar potency against wild-type or mutant ER, e.g., by inducing degradation of the wild-type or mutant ER as a selective estrogen receptor degrader (SERD).

In some embodiments of the disclosed methods, a subject in need thereof has one or more mutations in ESR1, which encodes the ER receptor alpha. The amino acid sequence of human estrogen receptor isoform 1 is as follows:

```
SEQ ID NO 1:
    1 MTMTLHTKAS GMALLHQIQG NELEPLNRPQ
        LKIPLERPLG EVYLDSSKPA VYNYPEGAAY
  61 EFNAAAAANA QVYGQTGLPY GPGSEAAAFG
        SNGLGGFPPL NSVSPSPLML LHPPPQLSPF
121 LQPHGQQVPY YLENEPSGYT VREAGPPAFY
        RPNSDNRRQG GRERLASIND KGSMAMESAK
181 ETRYCAVCND YASGYHYGVW SCEGCKAFFK
        RSIQGHNDYM CPATNQCTID KNRRKSCQAC
241 RLRKCYEVGM MKGGIRKDRR GGRMLKHKRQ
        RDDGEGRGEV GSAGDMRAAN LWPSPLMIKR
301 SKKNSLALSL TADQMVSALL DAEPPILYSE
        YDPTRPFSEA SMMGLLINLA DRELVHMINW
361 AKRVPGFVDL TLHDQVHLLE CAWLEILMIG
        LVWRSMEHPG KLLFAPNLLL DRNQGKCVEG
421 MVEIFDMLLA TSSRERMMNL QGEEFVCLKS
        IILLNSGVYT FLSSILKSLE EKDHIHRVLD
481 KITDTLIHLM AKAGLTLQQQ HQRLAQLLLI
        LSHIRHMSNK GMEHLYSMKC KNVVPLYDLL
541 LEMLDAHRLH APTSRGGASV EETDQSHLAT
        AGSTSSHSLQ KYYITGEAEG FPATV
```

In some embodiments, the subject has one or more missense mutations in ESR1 selected from S47R, A65V, N69K, P147Q, G160D, P222S, R233G, K252N, R271K, E279V, S282C, A293V, K303R, S329Y, G344D, L370F, E380Q, V392I, M396V, N407I, V418E, G420D, M421V, S432L, M437I, G442R, F461V, S463P, L466Q, E471D, V478L, R503W, E523Q, H524L, K531E, N532K, V533M, V534E, P535H/T, L536H/Q/P/R, Y537C/D/H/N/S, D538G/N, L540Q, E542G, A546D/T, L549P, R555C/H, G557R, T570I, S576L, A593D, and combinations thereof. The subject may have ESR1 mutations on the same allele or different alleles. The subject may be heterozygous or homozygous for the ESR1 mutations.

In some embodiments of the disclosed methods, a subject has one or more ESR1 mutations prior to having been treated with any chemotherapeutic agent such as a SERD, an aromatase inhibitor (AI), or a selective estrogen receptor modulator (SERM) and may be characterized as having an ESR1 mutation de novo. In some embodiments, the ESR1 provides the breast cancer with resistance to anti-ER therapy (e.g., where the mutation provides resistance to treatment with a SERD, an AI, or a SERM). In some embodiments of the disclosed methods, a subject acquires one or more ESR1 mutations after having anti-ER therapy (e.g., after anti-ER therapy with a SERD, an AI, or a SERM and the acquired mutation provides the cancer with resistance to treatment with the SERD, the AI, or the SERM).

The term “ER+ breast cancer” as used herein refers to breast cancers that express wild-type or mutant ER. ER+ breast cancers may require activation of the wild-type or mutant ER by estrogen as a ligand in order to proliferate. ER+ breast cancers may express a mutant ER that is constitutively active and does not require estrogen as a ligand for activity.

### Formulations and Dosages

In some embodiments of the disclosed methods, imlunestrant is administered as part of a pharmaceutical formulation. In some embodiments, the pharmaceutical formulation is a solid formulation. The formulation including imlunestrant, or a pharmaceutically acceptable salt, amorphous form, or polymorph form thereof, can be administered by a variety of routes, depending upon the treatment desired and upon the area to be treated. In some embodiments, administration is oral. Oral administration can include a dosage form formulated for once-daily or twice-daily (BID) administration.

The compositions disclosed herein may comprise as an active pharmaceutical ingredient imlunestrant or a pharmaceutically acceptable salt, amorphous form, or polymorph form thereof. The compositions comprising imlunestrant or a pharmaceutically acceptable salt, amorphous form, or polymorph form thereof can be formulated in a unit dosage form, each dosage containing from about 1 to about 1,000 mg (1 g), more usually about 200 mg to about 400 mg, of the active ingredient. The term “unit dosage form” refers to physically discrete units suitable as unitary dosages for human subjects and other patients, each unit containing a predetermined quantity of active material (i.e., imlunestrant or a pharmaceutically acceptable salt, amorphous form, or polymorph form thereof) calculated to produce the desired therapeutic effect, in association with a suitable pharmaceutical excipient.

In some embodiments, the solid formulation is formulated as a 100 mg, 200 mg, 300 mg, or 400 mg dosage form. In some embodiments, the solid formulation is formulated as a 200 mg dosage form. In some embodiments, the solid formulation is formulated as a 400 mg dosage form. In some embodiments, the dosage form refers to the free base of imlunestrant in the solid formulation. In some embodiments, the dosage form refers to a pharmaceutically acceptable salt of imlunestrant in the solid formulation. In some embodiments, dosage forms of pharmaceutically acceptable salts of imlunestrant may include imlunestrant tosylate salt or imlunestrant 4-methylbenzenesulfonic acid salt.

In some embodiments, the compositions provided herein contain from about 1 to about 1,000 mg (1 g), more usually about 200 mg to about 400 mg, of the active ingredient. One having ordinary skill in the art will appreciate that this embodies compounds or compositions containing about 100 mg to about 400 mg, 100 mg to about 300 mg, 100 mg to about 200 mg, or 200 mg to about 400 mg, of the active ingredient, which may include a free base of imlunestrant or a pharmaceutically acceptable salt of imlunestrant.

The daily dosage of the compound of imlunestrant or a pharmaceutically acceptable salt, amorphous form, or polymorph form thereof can be varied over a wide range from 1.0 to 10,000 mg per adult human per day, or higher, or any range therein. In some embodiments, for oral administration, the compositions can be provided in the form of tablets containing about 100, 200, 300, or 400 milligrams of the active ingredient for the symptomatic adjustment of the dosage to the patient to be treated.

An effective amount of the drug is ordinarily supplied at a dosage level of from about 0.1 mg/kg to about 1000 mg/kg of body weight per day, or any range therein. In some embodiments, the range is from about 0.5 to about 500 mg/kg of body weight per day, or any range therein. In some embodiments, from about 1.0 to about 250 mg/kg of body weight per day, or any range therein. In some embodiments, from about 0.1 to about 100 mg/kg of body weight per day, or any range therein. In some embodiments, the range is from about 0.1 to about 50.0 mg/kg of body weight per day, or any amount or range therein. In some embodiments, the range is from about 0.1 to about 15.0 mg/kg of body weight per day, or about 0.5 mg/kg to about 10 mg/kg, or 1 mg/kg to about 9 mg/kg, or about 2 mg/to about 8 mg/kg or about 3 mg/kg to 7 mg/kg. Additionally, the imlunestrant can be administered, for example, in an amount of about 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4, mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, etc.

In one aspect, the subject is administered about 0.1 mg/kg to about 15.0 mg/kg of body weight per day of imlunestrant or a pharmaceutically acceptable salt, amorphous form, or polymorph form, thereof, or a composition comprising imlunestrant or a pharmaceutically acceptable salt, amorphous form, or polymorph form, thereof.

Pharmaceutical compositions containing imlunestrant or a pharmaceutically acceptable salt, amorphous form, or polymorph form thereof can be administered on a regimen of 1 to 4 times per day. More preferably, the subject is administered imlunestrant or a pharmaceutically acceptable salt, amorphous form, or polymorph form, thereof, or a composition comprising imlunestrant or a pharmaceutically acceptable salt, amorphous form, or polymorph form thereof, once or twice daily, or in a single daily dose. A single daily dose may be defined as a single dose within a 24 hour period. Daily administration can be once-daily or in multiple doses, e.g., twice-daily (BID) administration. In some aspects, the imlunestrant or a pharmaceutically acceptable salt thereof, is a polymorph. Alternatively, the imlunestrant or a pharmaceutically acceptable salt thereof, is amorphous. In certain aspects, the imlunestrant is the free amine.

The active compound may be effective over a wide dosage range and is generally administered in a pharmaceutically effective amount. Optimal dosages to be administered can be readily determined by those skilled in the art. It will be understood, therefore, that the amount of the compound actually administered will usually be determined by a physician, and will vary according to the relevant circumstances, including the mode of administration, the actual compound administered, the strength of the preparation, the condition to be treated, and the advancement of the disease condition. In addition, factors associated with the particular patient being treated, including; patient response, age, weight, diet, time of administration and severity of the patient's symptoms, will result in the need to adjust dosages.

### Polymorphic Forms of Imlunestrant

In some embodiments, the solid formulations disclosed herein include a polymorph form of the compound of imlunestrant. The forms include, e.g., free bases, solvates, hydrates, salts, and non-solvated forms of imlunestrant, including, for example, polymorph forms as disclosed in U.S. Pat. Nos. 10,654,866, 11,117,902, and US Publication No. 20210403480, which are incorporated herein by reference in their entireties. In some embodiments, the polymorph form of imlunestrant is a pharmaceutically acceptable salt. In some embodiments, the polymorph form of imlunestrant is a free base. In some embodiments, the polymorph form of imlunestrant is a crystalline form.

### Salts of Imlunestrant

In some embodiments, the imlunestrant administered in the disclosed methods is a pharmaceutically acceptable salt. In some embodiments, a pharmaceutically acceptable salt of imlunestrant is a 4-methylbenzenesulfonic acid salt. In some embodiments, a pharmaceutically acceptable salt of imlunestrant is a tosylate salt. Salts of imlunestrant as well as methods for preparing such salts, are described in U.S. Pat. Nos. 10,654,866, 11,117,902, and US Publication No. 20210403480, which are incorporated herein by reference in their entireties.

### Methods for Preparing Imlunestrant

In some embodiments, imlunestrant may be obtained from a commercial source or, in the alternative, imlunestrant may be prepared by synthetic methods such as those disclosed and described elsewhere (e.g., U.S. Pat. Nos. 10,654,866, 11,117,902, and US Publication No. 20210403480).

### Methods of Administering Imlunestrant

In some embodiments of the disclosed methods, imlunestrant may be administered by methods as disclosed in U.S. Pat. Nos. 10,654,866, 11,117,902, US Publication No. 20210403480, and US Publication No. 20220288019, which are incorporated herein by reference in their entireties. In some embodiments, the imlunestrant may be administered to a subject having cancer at a dose between about 200 mg and about 400 mg at least once a day for at least one week. In some embodiments, the imlunestrant may be administered to a subject having cancer at a dose of about 200 mg, about 300 mg, or about 400 mg. In some embodiments, the imlunestrant may be administered to a subject having cancer at a dose of about 400 mg. In some embodiments of the disclosed methods, imlunestrant may be administered as a first line treatment, e.g., where the subject is naïve to treatment with any previous chemotherapeutic agent for treating cancer. In some embodiments, imlunestrant may be administered as a second line treatment, e.g., where the subject previously was administered a chemotherapeutic treatment for cancer prior to imlunestrant being administered to the subject.

### Methods of Administering Imlunestrant and Additional Pharmaceutical Agents

In some embodiments of the disclosed methods, a subject is administered imlunestrant and further is administered one or more additional pharmaceutical agents. In some embodiments, the subject is administered imlunestrant and is administered a second therapeutic agent in simultaneous, separate, or sequential combination. In some embodiments, the subject has been administered a therapeutic agent other than imlunestrant, prior to the subject being administered imlunestrant, and the subject subsequently is administered imlunestrant. Methods for administered imlunestrant and one or more additional therapeutic agents are disclosed in US Publication No. 20220288019, which is incorporated herein by reference in its entirety. Therapeutic agents administered in simultaneous, separate, or sequential combination with imlunestrant may include CDK 4/6 inhibitors, PI3K inhibitors, aromatase inhibitors (AIs) (e.g., anastrozole, exemestane, and letrozole), mTOR inhibitors (e.g., everolimus), anti-HER2 biologics (e.g., trastuzumab, fam-trastuzumab deruxtecan-nxki, and pertuzumab. Additional therapeutic agents may include antidiarrheal agents. The additional pharmaceutical agents may be administered according to the indication on their respective labels or as known in the art.

In some embodiments of the disclosed methods, the additional therapeutic agent is a CDK 4/6 inhibitor, a PI3K inhibitor, or a combination thereof. CDK 4/6 inhibitors may include palbociclib, ribociclib, abemaciclib, dalpiciclib, trilaciclib, auceliciclib, BEBT-209, BPI-16350, CS-3002, ETH-155008, FCN-437c, GLR-2007, HS-10342, 1-022, lerociclib, narazaciclib, PRT-3645, SHR-6390, TQB-3616, TY-302, XH-30002, XZP-3287, and combinations thereof. The CDK 4/6 inhibitors may be administered according to the indication on their respective labels or as known in the art. Suitable PI3K inhibitors may include selective PI3K alpha inhibitors. Suitable PI3K inhibitors may include alpelisib (BYL-719), buparlisib (BKM120), copanlisib (BAY 80-6946), duvelisib, idelalisib, linperlisib, parasaclisib, ACP-319, amdizalisib, ART-001, BGB-10188, BPI-21668, BR-101801, buparlisib, CHF-6523, CT-365, CYH33, eganelisib, HEC-89736PTSA 0.5H2O, HH-CYH33, HS-10352, inavolisib (GDC-0077), leniolisib, LOXO-783, MEN-1611, NPT520-34, RLY-2608, roginolisib, serabelisib, SHC-01478M, ST-814, tenalisib, TOS-358, TQB-3525, umbralisib, zandelisib, ZX-101, ZX-4081, and combinations thereof. Suitable PI3K inhibitors may include PI3K inhibitors disclosed in one or more of the following published patent applications: WO2021055747, WO2021202964, WO2021222556, WO2022198024, WO2022235574, WO2022235575, WO2022251482, WO2022265993, WO2023018636, WO2023039532, WO2023056407, WO2023060262, WO2023069644, WO2023078401, WO2023081209, WO2023081757, WO2023081759, WO2023104111, WO2023159155, WO2023173124, WO2023192416, WO2023212693, WO2023220131, WO2023230262, WO2023288242, WO2024008122, WO2024026423, WO2024030863, WO2024054469, WO2024064024, WO2024086296, WO2024086664, and WO2024086789. The PI3K inhibitors may be administered according to the indication on their respective labels or as known in the art.

In some embodiments of the disclosed methods, the subject is CDK4/6 inhibitor naïve prior to administering to the subject imlunestrant of the additional pharmaceutical agent. In some embodiments of the disclosed methods, the subject is PI3K inhibitor naïve prior to administering to the subject imlunestrant of the additional pharmaceutical agent.

### Subjects of the Disclosed Treatment Methods

In some embodiments of the disclosed methods, a suitable subject is a subject having cancer. In some embodiments, the subject has breast cancer. In some embodiments, a suitable subject has metastatic breast cancer (mBC). In some embodiments, the subject has advanced breast cancer. In some embodiments, the subject has early breast cancer. In some embodiments, the subject has breast cancer that has metastasized to the brain of the subject. In some embodiments, the subject has breast cancer and is at risk for developing breast cancer that has metastasized to the brain of the subject. In some embodiments of the disclosed methods the subject has ER+ breast cancer. In some embodiments, the subject is at risk for developing ER+ breast cancer, for example, where the subject has ER− cancer and the subject is at risk for developing ER+ breast cancer. In some embodiments, the subject has HER− cancer. In some embodiments, the subject has HER+ breast cancer. In some embodiments, the subject is at risk for developing HER− breast cancer, for example where the subject has HER2+ cancer and is at risk for developing HER2− breast cancer (e.g., where the subject is administered an anti-HER2 therapeutic). In some embodiments, the subject is at risk for developing HER2+ breast cancer, for example where the subject has HER2− and is at risk for developing HER2+ breast cancer. In some embodiments of the disclosed methods, suitable subject for the disclosed methods may include subjects having or at risk for developing ER+/HER2+ breast cancer or ER+/HER2− breast cancer.

Subjects that have ER+ breast cancer may express a wild-type ER protein, a mutant ER protein, or both (e.g., where one allele expresses the wild-type ER protein and the other allele expresses the mutant ER protein). Subject that ER+ breast cancer may express a mutant protein having one or more mutations selected from S47R, A65V, N69K, P147Q, G160D, P222S, R233G, K252N, R271K, E279V, S282C, A293V, K303R, S329Y, G344D, L370F, E380Q, V392I, M396V, N407I, V418E, G420D, M421V, S432L, M437I, G442R, F461V, S463P, L466Q, E471D, V478L, R503W, E523Q, H524L, K531E, N532K, V533M, V534E, P535H/T, L536H/Q/P/R, Y537C/D/H/N/S, D538G/N, L540Q, E542G, A546D/T, L549P, R555C/H, G557R, T570I, S576L, A593D, and combinations thereof.

In the disclosed methods, prior to administering imlunestrant to the subject, the subject may be identified as a subject having an ER+ cancer that expresses wild-type ER, mutant ER, or both. the subject may be identified as a subject having an ER+ cancer that expresses wild-type ER, mutant ER, or both by methods that include immunoassays, nucleic acid assays, or both, as known in the art.

### ILLUSTRATIVE EMBODIMENTS

The following embodiments provide an illustration of some of the aspects and embodiments described above, and are not intended to limit the scope of the claimed invention.

Embodiment 1. A method for preventing brain metastases in a subject having a cancer, comprising administering to the subject in need of prevention, imlunestrant or a pharmaceutically acceptable salt thereof, or a composition comprising imlunestrant or a pharmaceutically acceptable salt thereof in an amount effective to prevent brain metastases in the subject.

Embodiment 2. A method of reducing the risk of occurrence or development of brain metastases in a subject having a cancer, comprising administering to the subject in need thereof, imlunestrant or a pharmaceutically acceptable salt thereof, or a composition comprising imlunestrant or a pharmaceutically acceptable salt thereof in an amount effective to reduce the risk of occurrence of brain metastases in the subject.

Embodiment 3. A method of treating cancer that reduces the risk of developing detectable metastases of the cancer to the brain, comprising administering to the subject having the cancer, imlunestrant or a pharmaceutically acceptable salt thereof, or a composition comprising imlunestrant or a pharmaceutically acceptable salt thereof in an amount effective to reduce the risk of detectable metastases in the brain of the subject.

Embodiment 4. A method for delaying onset of brain metastases in a subject having a cancer, comprising administering to the subject in need thereof, imlunestrant or a pharmaceutically acceptable salt thereof, or a composition comprising imlunestrant or a pharmaceutically acceptable salt thereof in an amount effective to delay onset of brain metastases in the subject.

Embodiment 5. A method of preventing onset of brain metastases in a subject having a cancer, comprising administering to the subject in need thereof, imlunestrant or a pharmaceutically acceptable salt thereof, or a composition comprising imlunestrant or a pharmaceutically acceptable salt thereof in an amount effective to prevent onset of brain metastases in the subject.

Embodiment 6. A method for inhibiting metastatic progression of cancer in the brain of a subject having a cancer, comprising administering to the subject in need thereof, imlunestrant or a pharmaceutically acceptable salt thereof, or a composition comprising imlunestrant or a pharmaceutically acceptable salt thereof in an amount effective to inhibit metastatic progression of cancer in the brain of the subject.

Embodiment 7. A method for inhibiting growth and/or survival of metastatic cancer cells in the brain of a subject having a cancer, comprising administering to the subject in need thereof, imlunestrant or a pharmaceutically acceptable salt thereof, or a composition comprising imlunestrant or a pharmaceutically acceptable salt thereof in an amount effective to inhibit growth and/or survival of metastatic cancer cells in the brain of the subject.

Embodiment 8. A method for inhibiting cancer from progressing to metastatic brain cancer in a subject having a cancer, comprising administering to the subject in need thereof, imlunestrant or a pharmaceutically acceptable salt thereof, or a composition comprising imlunestrant or a pharmaceutically acceptable salt thereof in an amount effective to inhibit progression of the cancer to metastatic brain cancer in the subject.

Embodiment 9. A method for reducing the risk of developing, or the onset of, detectable brain metastases in a subject who has cancer, the method comprising administering to the subject in need thereof, imlunestrant or a pharmaceutically acceptable salt thereof, or a composition comprising imlunestrant or a pharmaceutically acceptable salt thereof in an amount effective to reduce the risk of developing, or the risk of onset of, detectable brain metastases in the subject.

Embodiment 10. A method for lengthening the period of survival of a subject having a cancer comprising administering to the subject in need thereof, imlunestrant or a pharmaceutically acceptable salt thereof, or a composition comprising imlunestrant or a pharmaceutically acceptable salt thereof in an amount effective to lengthen the period of survival of the subject, relative to the period of survival of the subject in the absence of administration.

Embodiment 11. The method of any one of embodiments 1-10, wherein the cancer is ER+ cancer.

Embodiment 12. The method of any one of embodiments 1-11, wherein the cancer is ER+ breast cancer.

Embodiment 13. The method of embodiment 12, wherein the cancer is ER+/HER2+ breast cancer.

Embodiment 14. The method of embodiment 12, wherein the cancer is ER+/HER2− breast cancer.

Embodiment 15. The method of embodiment 12, wherein the cancer expresses the wild-type ER protein.

Embodiment 16. The method of embodiment 12, wherein the cancer expresses a mutant ER protein.

Embodiment 17. The method of embodiment 16, wherein the mutant ER protein comprises one or more mutations selected from S47R, A65V, N69K, P147Q, G160D, P222S, R233G, K252N, R271K, E279V, S282C, A293V, K303R, S329Y, G344D, L370F, E380Q, V392I, M396V, N407I, V418E, G420D, M421V, S432L, M437I, G442R, F461V, S463P, L466Q, E471D, V478L, R503W, E523Q, H524L, K531E, N532K, V533M, V534E, P535H/T, L536H/Q/P/R, Y537C/D/H/N/S, D538G/N, L540Q, E542G, A546D/T, L549P, R555C/H, G557R, T570I, S576L, A593D, and combinations thereof.

Embodiment 18. The method of any one of embodiments 1-17, wherein the subject has metastatic breast cancer.

Embodiment 19. The method of embodiment 18, wherein the subject has brain metastatic breast cancer.

Embodiment 20. The method of embodiment 19, wherein the brain metastases comprise an undetected or an undetectable metastasis.

Embodiment 21. The method of any one of embodiments 1-17, wherein the subject has not had prior metastases.

Embodiment 22. The method of any one of embodiments 1-17, wherein the subject was previously treated for prior metastases.

Embodiment 23. The method of any one of embodiments 1-17, wherein the subject has a primary cancer associated with an increased risk and/or an increased likelihood of metastatic progression.

Embodiment 24. The method of any one of embodiments 1-17, wherein the subject was not known to have brain metastases prior to administering the imlunestrant or a pharmaceutically acceptable salt thereof, or a composition comprising imlunestrant or a pharmaceutically acceptable salt thereof.

Embodiment 25. The method of any one of embodiments 1-24, further comprising administering to the subject an additional anti-cancer agent in simultaneous, separate, or sequential combination with imlunestrant.

Embodiment 26. The method of embodiment 25, wherein the additional therapeutic agent is a CDK 4/6 inhibitor, a PI3K inhibitor, or a combination thereof.

Embodiment 27. The method of embodiment 26, wherein the CDK 4/6 inhibitor is selected from palbociclib, ribociclib, abemaciclib, dalpiciclib, trilaciclib, auceliciclib, BEBT-209, BPI-16350, CS-3002, ETH-155008, FCN-437c, GLR-2007, HS-10342, 1-022, lerociclib, narazaciclib, PRT-3645, TQB-3616, TY-302, XH-30002, XZP-3287, and combinations thereof.

Embodiment 28. The method of embodiment 26, wherein the CDK 4/6 inhibitor is selected from palbociclib, ribociclib, abemaciclib, dalpiciclib, trilaciclib, auceliciclib, lerociclib, and narazaciclib.

Embodiment 29. The method of embodiment 26, wherein the CDK 4/6 inhibitor is abemaciclib.

Embodiment 30. The method of any one of embodiment 1-18, wherein the subject is CDK4/6 inhibitor naïve.

Embodiment 31. The method of embodiment 26, wherein the PI3K inhibitor is selected from alpelisib, copanlisib, duvelisib, idelalisib, linperlisib, parasaclisib, ACP-319, amdizalisib, ART-001, BGB-10188, BPI-21668, BR-101801, buparlisib, CHF-6523, CT-365, eganelisib, HEC-89736PTSA 0.5H2O, HH-CYH33, HS-10352, inavolisib, leniolisib, LOXO-783, MEN-1611, NPT520-34, RLY-2608, roginolisib, serabelisib, SHC-01478M, tenalisib, TQB-3525, umbralisib, zandelisib, ZX-101, ZX-4081, and combinations thereof.

Embodiment 32. The method of embodiment 26, wherein the PI3K inhibitor is a selective PI3K alpha inhibitor.

Embodiment 33. The method of embodiment 32, wherein the selective Pi3K alpha inhibitor is alpelisib, BPI-21668, HH-CYH33, Inavolisib, LOXO-783, RLY-2608, or serabelisib.

Embodiment 33. The method of embodiment 32, wherein the selective PI3K alpha inhibitor is LOXO-783.

Embodiment 34. The method of embodiment 32, wherein the selective PI3K alpha inhibitor is alpelisib.

Embodiment 35. The method of any one of embodiment 1-34, wherein the patient is Pi3K inhibitor naïve.

Embodiment 36. The method of any one of embodiments 1-35, wherein prior to administering imlunestrant to the subject, the subject is identified by detecting wild-type ER expression or mutant ER expression.

Embodiment 37. The method of any one of embodiments 1-36, wherein the subject is administered about 0.1 mg/kg to about 15.0 mg/kg of body weight per day of imlunestrant or a pharmaceutically acceptable salt thereof, or a composition comprising imlunestrant or a pharmaceutically acceptable salt thereof.

Embodiment 38. The method of any one of embodiments 1-37, wherein when the subject is administered a dose of imlunestrant of about 200 mg to about 400 mg, once or twice a day, for at least about a week.

Embodiment 39. The method of any one of embodiments 1-38, wherein the imlunestrant is administered as a pharmaceutically acceptable salt which is a 4-methylbenzenesulfonic acid salt.

Embodiment 40. The method of any one of embodiments 1-38, wherein the imlunestrant is administered as a free base.

Embodiment 41. Imlunestrant or a pharmaceutically acceptable salt thereof, for use in the prevention of brain metastases in a subject who has cancer.

Embodiment 42. Imlunestrant or a pharmaceutically acceptable salt thereof, for use in reducing the risk of occurrence or development of brain metastases in a subject who has cancer.

Embodiment 43. Imlunestrant or a pharmaceutically acceptable salt thereof, for use in reducing the risk of developing detectable brain metastases in a subject who has cancer.

Embodiment 44. Imlunestrant or a pharmaceutically acceptable salt thereof, for use in delaying onset of brain metastases in a subject who has cancer.

Embodiment 45. Imlunestrant or a pharmaceutically acceptable salt thereof, for use in preventing onset of brain metastases in a subject who has cancer.

Embodiment 46. Imlunestrant or a pharmaceutically acceptable salt thereof, for use in inhibiting metastatic progression of cancer in the brain of a subject who has cancer.

Embodiment 47. Imlunestrant or a pharmaceutically acceptable salt thereof, for use in inhibiting growth and/or survival of metastatic cancer cells in the brain of a subject who has cancer.

Embodiment 48. Imlunestrant or a pharmaceutically acceptable salt thereof, for use in inhibiting a cancer from progressing to metastatic brain cancer in a subject who has cancer.

Embodiment 49. Imlunestrant or a pharmaceutically acceptable salt thereof, for use in reducing the risk of developing, or the onset of, detectable brain metastases in a subject who has cancer.

Embodiment 50. Imlunestrant or a pharmaceutically acceptable salt thereof, for use in lengthening the period of survival of a subject who has cancer.

Embodiment 51. Imlunestrant or a pharmaceutically acceptable salt thereof, for use according to any one of embodiments 41-50, wherein the cancer is ER+ cancer.

Embodiment 52. Imlunestrant or a pharmaceutically acceptable salt thereof, for use according to any one of embodiments 41-50, wherein the cancer is ER+ breast cancer.

Embodiment 53. Imlunestrant or a pharmaceutically acceptable salt thereof, for use according to any one of embodiments 41-50, wherein the cancer is ER+/HER2+ breast cancer.

Embodiment 54. Imlunestrant or a pharmaceutically acceptable salt thereof, for use according to any one of embodiments 41-50, wherein the cancer is ER+/HER2− breast cancer.

Embodiment 55. Imlunestrant or a pharmaceutically acceptable salt thereof, for use according to any one of embodiments 41-54, wherein the cancer expresses the wild-type ER protein.

Embodiment 56. Imlunestrant or a pharmaceutically acceptable salt thereof, for use according to any one of embodiments 41-54, wherein the cancer expresses a mutant ER protein.

Embodiment 57. Imlunestrant or a pharmaceutically acceptable salt thereof, for use according to embodiment 56, wherein the mutant ER protein comprises one or more mutations selected from S47R, A65V, N69K, P147Q, G160D, P222S, R233G, K252N, R271K, E279V, S282C, A293V, K303R, S329Y, G344D, L370F, E380Q, V392I, M396V, N407I, V418E, G420D, M421V, S432L, M437I, G442R, F461V, S463P, L466Q, E471D, V478L, R503W, E523Q, H524L, K531E, N532K, V533M, V534E, P535H/T, L536H/Q/P/R, Y537C/D/H/N/S, D538G/N, L540Q, E542G, A546D/T, L549P, R555C/H, G557R, T570I, S576L, A593D, and combinations thereof.

Embodiment 58. Imlunestrant or a pharmaceutically acceptable salt thereof, for use according to any one of embodiments 41-57, wherein the subject has metastatic breast cancer.

Embodiment 59. Imlunestrant or a pharmaceutically acceptable salt thereof, for use according to any one of embodiments 41-58, wherein the subject has brain metastatic breast cancer.

Embodiment 60. Imlunestrant or a pharmaceutically acceptable salt thereof, for use according to any one of embodiments 41-59, wherein the brain metastases comprise an undetected or an undetectable metastasis.

Embodiment 61. Imlunestrant or a pharmaceutically acceptable salt thereof, for use according to any one of embodiments 41-58, wherein the subject has not had prior metastases.

Embodiment 62. Imlunestrant or a pharmaceutically acceptable salt thereof, for use according to any one of embodiments 41-58, wherein the subject was previously treated for prior metastases.

Embodiment 63. Imlunestrant or a pharmaceutically acceptable salt thereof, for use according to any one of embodiments 41-62, wherein the subject has a primary cancer associated with an increased risk and/or an increased likelihood of metastatic progression.

Embodiment 64. Imlunestrant or a pharmaceutically acceptable salt thereof, for use according to any one of embodiments 41-62, wherein the subject was not known to have brain metastases prior to administering the imlunestrant or a pharmaceutically acceptable salt thereof, or a composition comprising imlunestrant or a pharmaceutically acceptable salt thereof.

Embodiment 65. Imlunestrant or a pharmaceutically acceptable salt thereof, for use according to any one of embodiments 41-64, in simultaneous, separate or sequential combination with an additional anti-cancer agent.

Embodiment 66. Imlunestrant or a pharmaceutically acceptable salt thereof, for use according to embodiment 65, wherein the additional therapeutic agent is a CDK 4/6 inhibitor, a PI3K inhibitor, or a combination thereof.

Embodiment 67. Imlunestrant or a pharmaceutically acceptable salt thereof, for use according to embodiment 66, wherein the CDK 4/6 inhibitor is selected from palbociclib, ribociclib, abemaciclib, dalpiciclib, trilaciclib, auceliciclib, BEBT-209, BPI-16350, CS-3002, ETH-155008, FCN-437c, GLR-2007, HS-10342, 1-022, lerociclib, narazaciclib, PRT-3645, TQB-3616, TY-302, XH-30002, XZP-3287, and combinations thereof.

Embodiment 68. Imlunestrant or a pharmaceutically acceptable salt thereof, for use according to embodiment 66, wherein the CDK 4/6 inhibitor is selected from palbociclib, ribociclib, abemaciclib, dalpiciclib, trilaciclib, auceliciclib, lerociclib, and narazaciclib.

Embodiment 69. Imlunestrant or a pharmaceutically acceptable salt thereof, for use according to embodiment 66, wherein the CDK 4/6 inhibitor is abemaciclib.

Embodiment 70. Imlunestrant or a pharmaceutically acceptable salt thereof, for use according to embodiment 41-69, wherein the subject is CDK4/6 inhibitor naïve.

Embodiment 71. Imlunestrant or a pharmaceutically acceptable salt thereof, for use according to 66, wherein the PI3K inhibitor is selected from alpelisib, copanlisib, duvelisib, idelalisib, linperlisib, parasaclisib, ACP-319, amdizalisib, ART-001, BGB-10188, BPI-21668, BR-101801, buparlisib, CHF-6523, CT-365, eganelisib, HEC-89736PTSA 0.5H2O, HH-CYH33, HS-10352, inavolisib, leniolisib, LOXO-783, MEN-1611, NPT520-34, RLY-2608, roginolisib, serabelisib, SHC-01478M, tenalisib, TQB-3525, umbralisib, zandelisib, ZX-101, ZX-4081, and combinations thereof.

Embodiment 72. Imlunestrant or a pharmaceutically acceptable salt thereof, for use according to embodiment 66, wherein the PI3K inhibitor is a selective PI3K alpha inhibitor.

Embodiment 73. Imlunestrant or a pharmaceutically acceptable salt thereof, for use according to embodiment 72, wherein the selective Pi3K alpha inhibitor is alpelisib, BPI-21668, HH-CYH33, Inavolisib, LOXO-783, RLY-2608, or serabelisib.

Embodiment 74. Imlunestrant or a pharmaceutically acceptable salt thereof, for use according to embodiment 72, wherein the selective PI3K alpha inhibitor is LOXO-783.

Embodiment 75. Imlunestrant or a pharmaceutically acceptable salt thereof, for use according to embodiment 72, wherein the selective PI3K alpha inhibitor is alpelisib.

Embodiment 76. Imlunestrant or a pharmaceutically acceptable salt thereof, for use according to any one of embodiment 41-75, wherein the patient is Pi3K inhibitor naïve.

Embodiment 77. Imlunestrant or a pharmaceutically acceptable salt thereof, for use according to any one of embodiments 41-76, wherein prior to administering imlunestrant to the subject, the subject is identified by detecting wild-type ER expression or mutant ER expression.

Embodiment 78. Imlunestrant or a pharmaceutically acceptable salt thereof, for use according to any one of embodiments 41-77, wherein the subject is administered about 0.1 mg/kg to about 15.0 mg/kg of body weight per day of imlunestrant or a pharmaceutically acceptable salt thereof, or a composition comprising imlunestrant or a pharmaceutically acceptable salt thereof.

Embodiment 79. Imlunestrant or a pharmaceutically acceptable salt thereof, for use according to any one of embodiments 41-78, wherein when the subject is administered a dose of imlunestrant of about 200 mg to about 400 mg, once or twice a day, for at least about a week.

Embodiment 80. Imlunestrant or a pharmaceutically acceptable salt thereof, for use according to any one of embodiments 41-79, wherein the subject is administered about 0.1 mg/kg to about 15.0 mg/kg of body weight per day of imlunestrant or a pharmaceutically acceptable salt thereof, or a composition comprising imlunestrant or a pharmaceutically acceptable salt thereof.

Embodiment 81. Imlunestrant or a pharmaceutically acceptable salt thereof, for use according to any one of embodiments 41-80, wherein when the subject is administered a dose of imlunestrant of about 200 mg to about 400 mg, once or twice a day, for at least about a week.

Embodiment 82. Imlunestrant or a pharmaceutically acceptable salt thereof, for use according to any one of embodiments 41-81, wherein the imlunestrant is administered as a pharmaceutically acceptable salt which is a 4-methylbenzenesulfonic acid salt.

Embodiment 83. Imlunestrant or a pharmaceutically acceptable salt thereof, for use according to any one of embodiments 41-81, wherein the imlunestrant is administered as a free base.

Embodiment 84. The method of any of claims 1-40 or the use of any of claims 41-83, wherein the method or use extends the subject's overall survival time (OS), for example, in comparison to a subject who was not administered imlunestrant or who was administered a SERD that is not imlunestrant.

Embodiment 85. The method of any of claims 1-40 or the use of any of claims 41-83, wherein the method or use extends the subject's progression-free survival (PFS) time, for example, in comparison to a subject who was not administered imlunestrant or who was administered a SERD that is not imlunestrant.

Embodiment 86. The method of any of claims 1-40 or the use of any of claims 41-83, wherein the method or use extends the subject's time to progression (TPP), for example, in comparison to a subject who was not administered imlunestrant or who was administered a SERD that is not imlunestrant.

Embodiment 87. The method of any of claims 1-40 or the use of any of claim 41-83, wherein the method or use extends the subject's disease-free survival time (DFS), for example, in comparison to a subject who was not administered imlunestrant or who was administered a SERD that is not imlunestrant.

Embodiment 88. The method of any of claims 1-40 or the use of any of claims 41-83, wherein the method or use extends the subject's event-free survival time (EFS), for example, in comparison to a subject who was not administered imlunestrant or who was administered a SERD that is not imlunestrant.

Embodiment 89. The method of any of claims 1-40 or the use of any of claims 41-83, wherein the method or use improves the subject's health-related quality of life (HRQoL) for example, in comparison to a subject who was not administered imlunestrant or who was administered a SERD that is not imlunestrant.

### EXAMPLES

The following examples provide an illustration of some of the aspects and embodiments described above, and are not intended to limit the scope of the claimed invention.

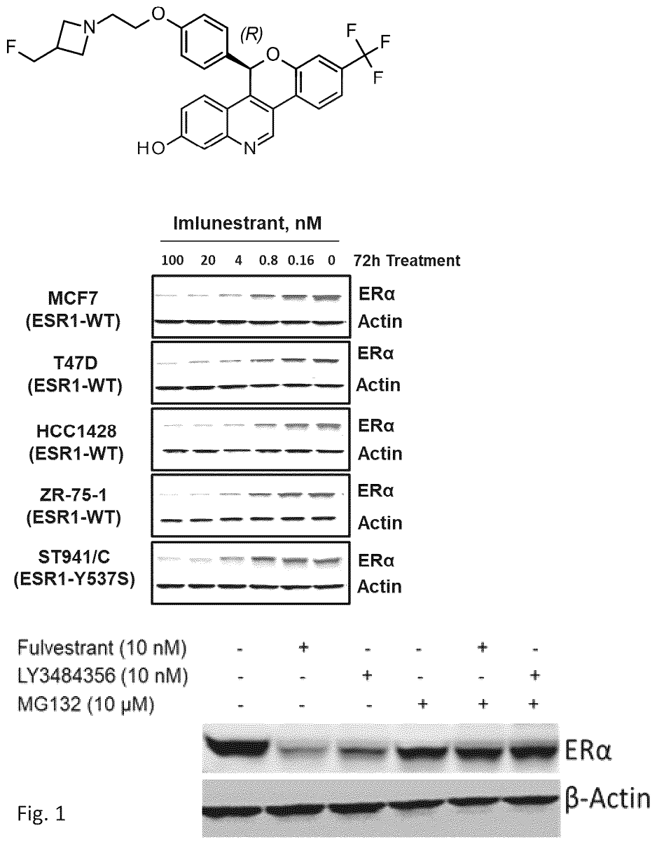

### Example 1. ERα Degradation Assay by Western Blot (FIG. 1)

MCF7, HCC1428, ZR-75-1, and T47D cells were obtained from ATCC. ST941/C cell line was licensed from START (South Texas Accelerated Research Therapeutics, LCC) and was derived from the ST941 breast START model. Cells were cultured in RPMI1640 (Gibco, 22400) with 10% HI FBS (Gibco, 10082) and 1% Anti-Anti (100×) (Gibco, 15240). Cells were plated in 6-well plates at a density of 0.45×10⁵ (ZR-75-1), 0.55×10⁵ (HCC1428), or 0.7×10⁵ (MCF7, ST941/C and T47D) cells in 2 mL of assay medium (Phenol-red free RPMI1640, Gibco, 11835), 10% charcoal stripped FBS (Gibco, 12676), and 1% Anti-Anti (100×) (Gibco, 15240)).

Cells were allowed to attach overnight before compound treatment. Cells were then treated with imlunestrant with a final DMSO concentration of 0.2% (v/v) in assay medium. A total of 5 concentrations were evaluated starting from 100 nM using a 1:5 dilution scheme. After 72-hour treatment, cells were washed with cold dPBS (Hyclone, SH30028) and then lysed using RIPA Buffer (Sigma, R0278) supplemented with Halt Phosphatase and Protease Inhibitor Cocktail (Thermo, 78441). Protein concentrations were determined using BCA Assay (Thermo, 23225) following the manufacturer's guidance. Four to twenty percent Criterion Tris-HCl precast gels (Bio-Rad, 345-0034) were loaded with 5 to 10 μg of total protein per well, electrophoresed, then protein was transferred with the Trans-Blot Turbo Midi Nitrocellulose Transfer Packs (Bio-Rad, 170-4159). Membranes were blocked with SEA BLOCK Blocking Buffer (Thermo, 37527) for 1 hour, and then incubated with primary antibodies ERα (Clone SP1) (Thermo, RM9101), PgR (Dako, M3569), or actin (Sigma A5441) overnight at 4° C. After thorough washing, the membrane was then incubated with secondary antibodies (Alexa Fluor®680 goat anti-rabbit (Life Technologies, A21076) or IRDye® 800CW goat anti-mouse (Li-Cor, 926-32210)) for 1 hour. The membrane was scanned using Li-Cor Odyssey Classic Infrared Imaging System at 700 and 800 nM.

Images were processed and analyzed using Image Studio Ver 3.1 (Li-Cor). MG132 cotreatment with fulvestrant or imlunestrant: Cells were treated with either 10 nM imlunestrant or fulvestrant, with or without 10 μM MG132 for 24 Hours. Western blot was performed as described for ERα degradation assay.

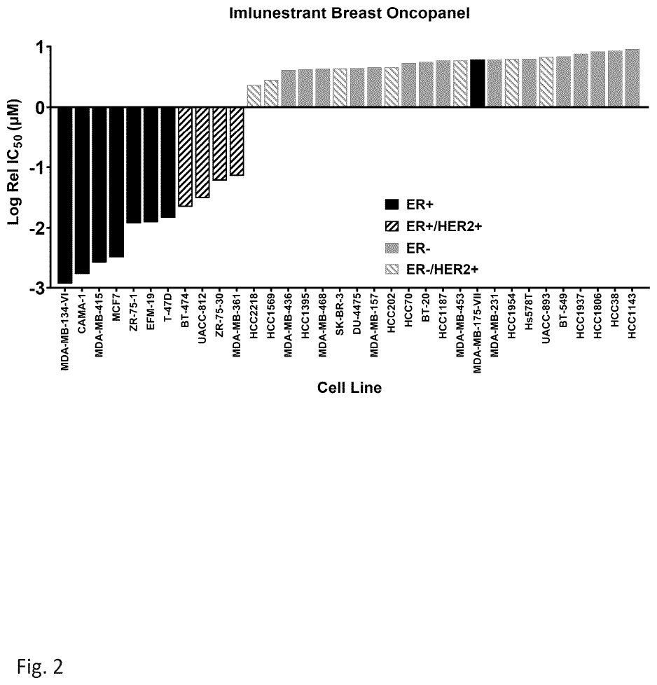

### Example 2. Anti-Proliferative Activity of Imlunestrant in a Panel of ER+ and ER− Breast Cancer Cell Lines (FIG. 2)

To examine the anti-proliferative activity of imlunestrant, a panel of ER+ and ER-breast cancer cell lines (All from ATCC) were plated in 384-well plates in the vendor recommended medium and allowed to attach overnight. Cells were then treated for two doubling times with 10 serial dilutions of imlunestrant starting at 20 μM with a 1:3 dilution scheme. Cells were fixed with 96% cold ethanol, then stained with propidium iodide (PI). Plates were scanned with an Acumen eX3 instrument and cell number was calculated with an Acumen algorithm. IC₅₀ values were determined by curve fitting the cell number data to a four-parameter logistic using GENE DATA™.

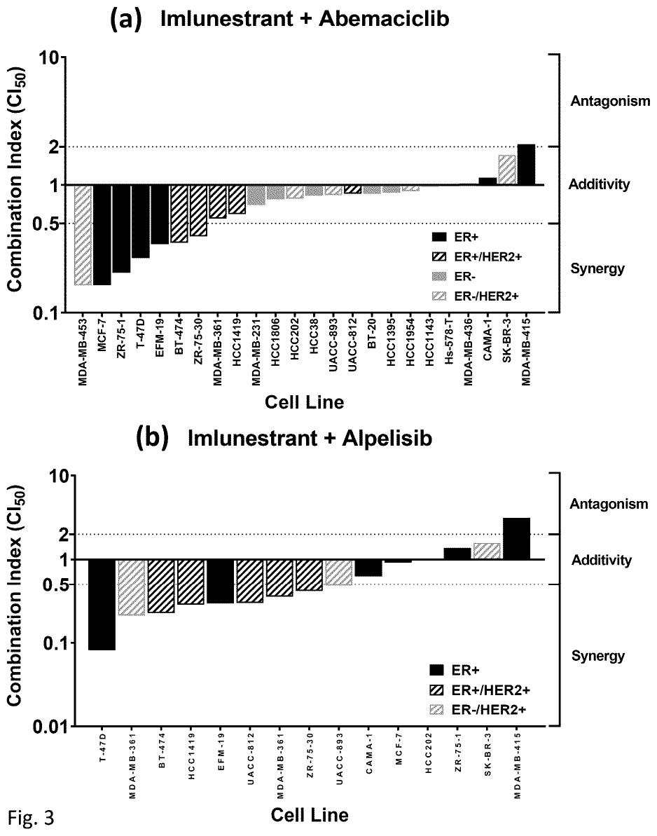

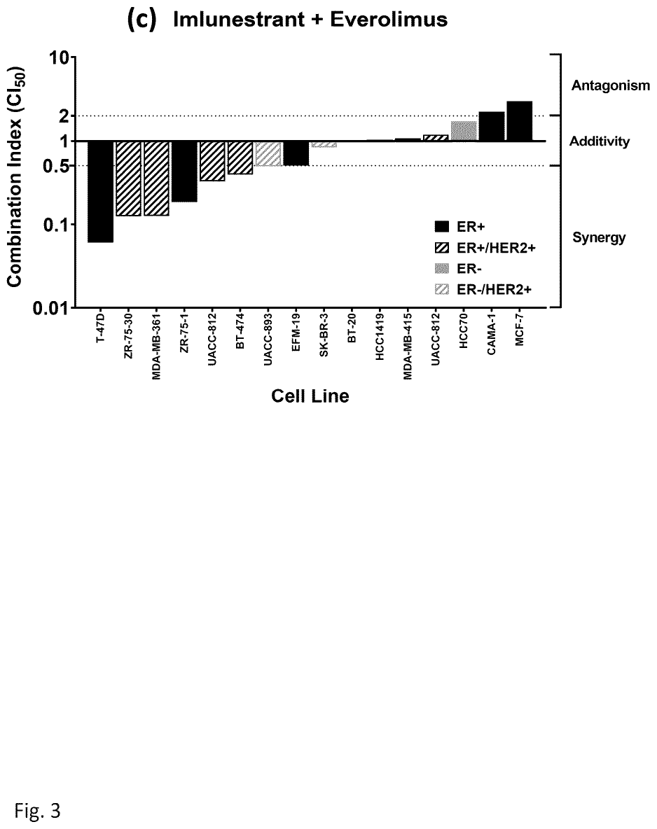

### Example 3. Combination Effect of Imlunestrant and Abemaciclib, Alpelisib, and Everolimus (FIG. 3)

Indicated cell lines (ATCC) were plated in 96-well plates, allowed to attach overnight, then treated with imlunestrant, abemaciclib, alpelisib, and everolimus as single agents and in combination with imlunestrant. For the 10-point combination treatment, each compound was tested in 10 single treatment concentrations (starting at 20 μM and then 1:3 dilution scheme) and in 10 combinatorial concentrations of two compounds (starting at 1 μM each and then 1:3 dilution scheme). Cells were grown for two doubling times then fixed with cold 96% ethanol and stained with a PI solution in PBS containing 50 ug/mL RNase. Staurosporine (2 μM) treatment was used for normalization. Plates were analyzed as described in Cell proliferation assay. The IC₅₀s were determined for single agent and combination treatment, which were used to calculate the combination index at 50% inhibition (CI₅₀). Biological synergy, additivity, and antagonism was determined as follows: CI₅₀<0.5=synergy, 2>CI₅₀>0.5=additivity, CI₅₀>2=antagonism.

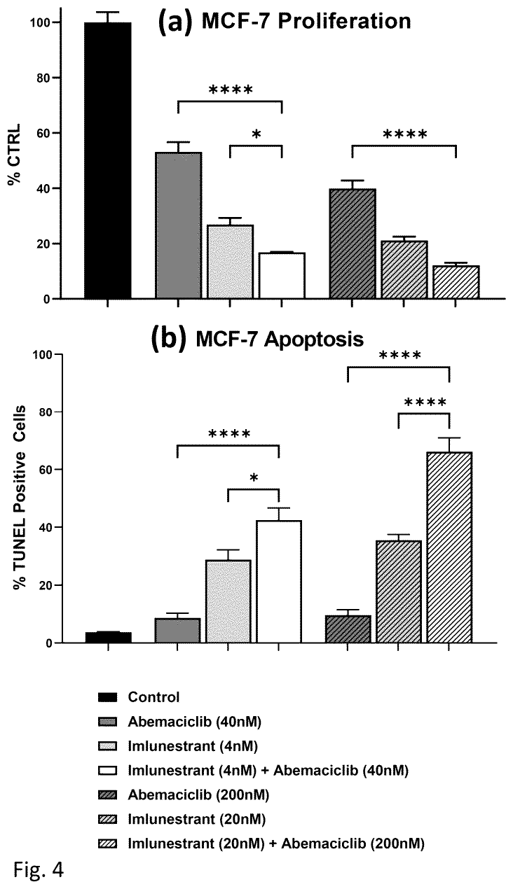

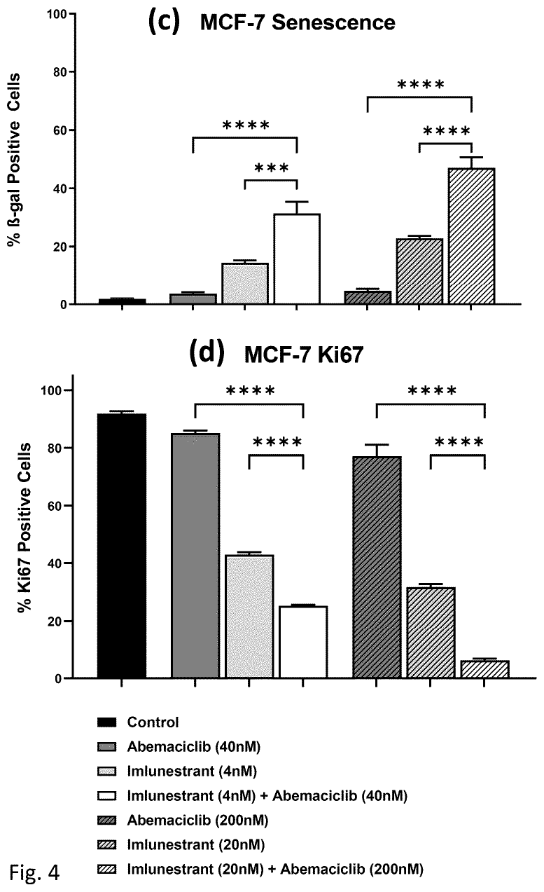

### Example 4. Effect and Mechanism of Action of Imlunestrant and Abemciclib, Alone and in Combination in MCF-7 Cells (FIG. 4)

MCF-7 cells were plated in 96-well plates, allowed to attached overnight and treated with the indicated drug or combination for 8 days. 1 μM nocodazole was used as the positive control for apoptosis and 0.5 μM adriamycin was used as positive control for senescence. Following treatment, cells were fixed with 3.7% paraformaldehyde, permeabilized with 0.1% Triton X-100 in PBS, and. blocked with 1% BSA in PBS. Cells were incubated with primary antibodies (K167 ABCAM, ab16667 or β-Gal ABCAM, ab12081) overnight at 4° C., followed by two washes with PBS and incubation with secondary antibody (Alexa Fluor®488 goat anti-rabbit, Invitrogen A11008; Alexa Fluor®647 goat anti-chicken, Abcam ab150171) for one hour at room temperature. Cells were washed three times with PBS. TUNEL reagent was added to wells without antibody according to manufacturer's protocol (Roche, 11767305001, 11767291910; Molecular Probes, C11397) and incubated for 3 hours at room temperature in the dark. Following 3 PBS washes, DNA was stained with DAPI (Sigma, D9564) diluted 1:1000 in PBS with 50 ug/mL RNAse. Plates were read with an Acumen eX3 instrument. Gates for percent responders (percent positive for desired marker) were set based on the DMSO-treated group and cell number was calculated with an Acumen algorithm.

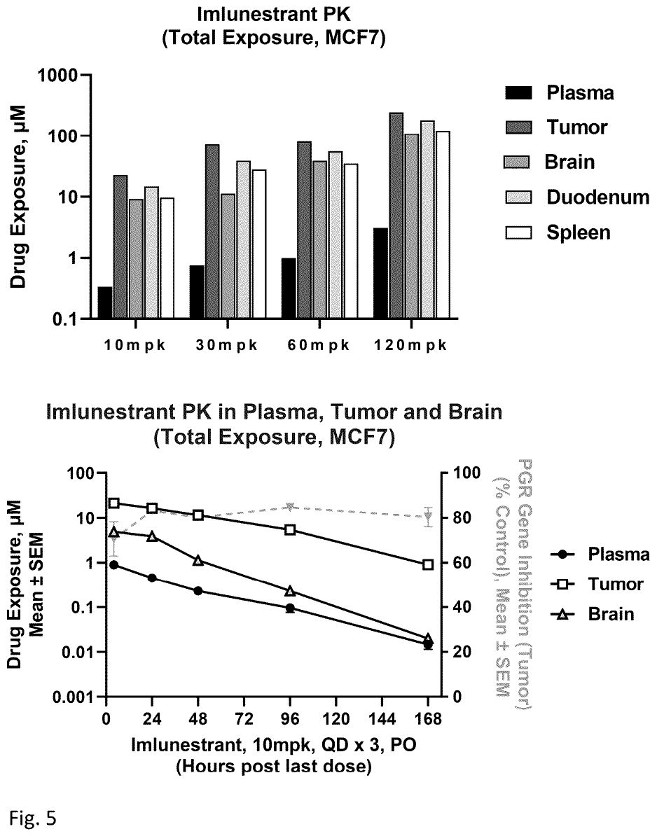

### Example 5. Drug Exposure (FIG. 5)

MCF-7 breast cancer cells were obtained from ATCC (HTB-22). The cells were maintained in growth media, MEM (Gibco, 11095-080), 0.1 mM MEM NEAA (Gibco, 11140 050), 1 mM sodium pyruvate (Gibco, 11360-070), and 10% FBS (Gibco, 10082). Imlunestrant was synthesized and was formulated in 1% hydroxyethylcellulose/0.25% Tween 80/0.05% Antifoam for oral administration by gavage. Approximately 5×10⁶ MCF-7 cells in HBSS were mixed with an equal volume of Matrigel (BD Bioscience, Frankline Lakes, NJ) and implanted subcutaneously into the flanks of the animals. When tumors reached an average size of 200 to 300 mm3, the animals were randomized into 5 animals per group. Imlunestrant was administered orally at 3, 10, or 30 mg/kg, once daily (QD) for three days. Animals were sacrificed using CO2 and cervical dislocation at 24 hours post last dose. Tumors, organs (brain, duodenum, spleen) and plasma were collected, snap frozen and sent to Q2 for drug exposure analysis.

To demonstrate the inhibition of ERα-mediated transcription and antagonistic activity in vivo, inhibition of PgR, a downstream transcriptional target of ERα, was measured in the tumors after 3 days of once daily 10 mg/kg oral dosing. Animals were sacrificed using CO2 and cervical dislocation at the following timepoints: 4, 12, 24, 36, 96, or 168 hours post-last dose. Xenograft tissues were collected and cut in half; one part of the tumor was frozen in RNALater solution (Thermo Fisher, AM7020) and the other part and the brain was snap frozen and sent to Q2 for drug exposure analysis.

Tumors that were collected in RNALater solution were cut into small pieces and then transferred to FastPrep lysing matrix A tube (MP Biomedicals) containing Buffer RLT with β-mercaptoethanol and processed according to manufacturer's protocols. Total RNA was then isolated using RNeasy Mini Kit (Qiagen, 74104) following the manufacturer's protocol. cDNA was generated using High-Capacity cDNA Reverse Transcription Kit (Applied Biosystem, 4368813). Quantitative real time PCR was performed in 384-well plates and thermal cycling reaction was carried out in an ABI HT7900 instrument. The final 10-μL PCR mixture contained 0.5 μL cDNA, 0.25 μL primer/probe set, and 5 μL 2× Absolute Blue qPCR ROX Mix (Thermo Scientific, AB-4139/A). Primer/probe sets were purchased from ThermoFishers Scientific (PGR: Hs01556701_m1 and GAPDH: Hs99999905_m1). Data were analyzed using Lilly Taqman Data Analysis tool. The housekeeping gene used to normalize results was GAPDH. Expression was calculated relative to the DMSO control for each sample.

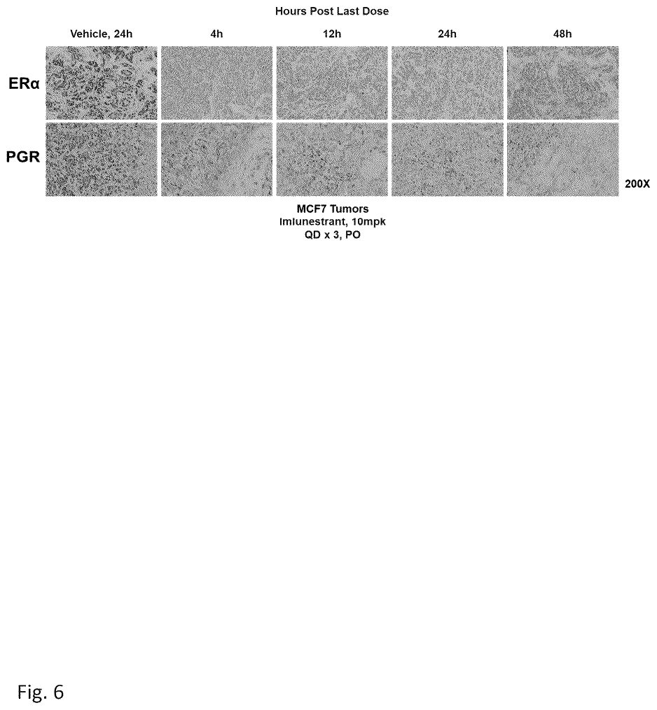

### Example 6. ERα and PgR Immunohistochemical Analysis (FIG. 6)

MCF7 tumors were implanted as described for drug exposure studies. After 3 days of once daily 10 mg/kg oral dosing, animals were sacrificed using CO2 and cervical dislocation at the following timepoints: 4, 12, 24, and 48 hours post last dose. Tumors were collected, fixed with 10% neutral buffered formalin, and embedded in paraffin blocks. Blocks were then transferred to ARUP laboratories for processing, staining and analysis (ERα, 2004516 and PgR, 2004525).

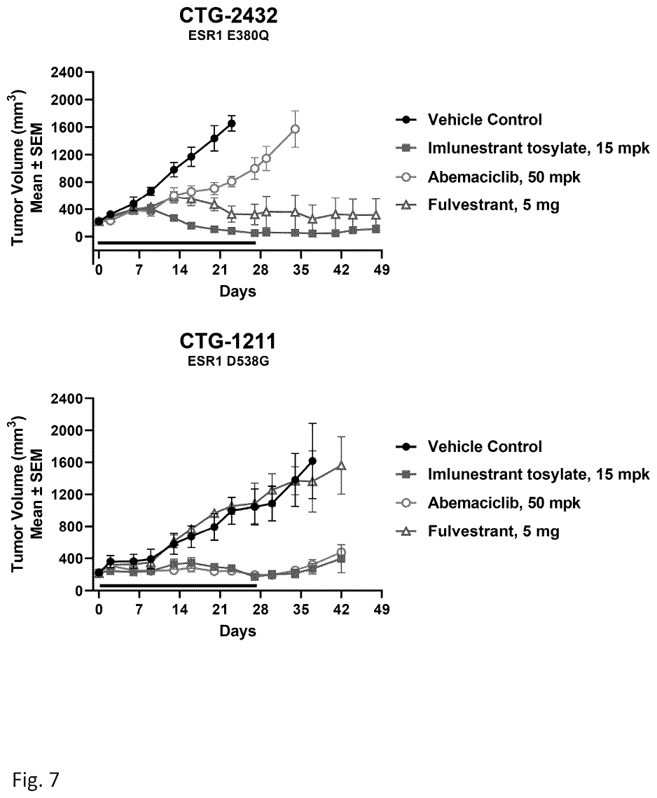

### Example 7. In Vivo Efficacy in a CTG-2432 (ESR1 E3800) Xenograft Model, and a CTG-1211 (ESR1 D538G) Xenograft Model (FIG. 7)

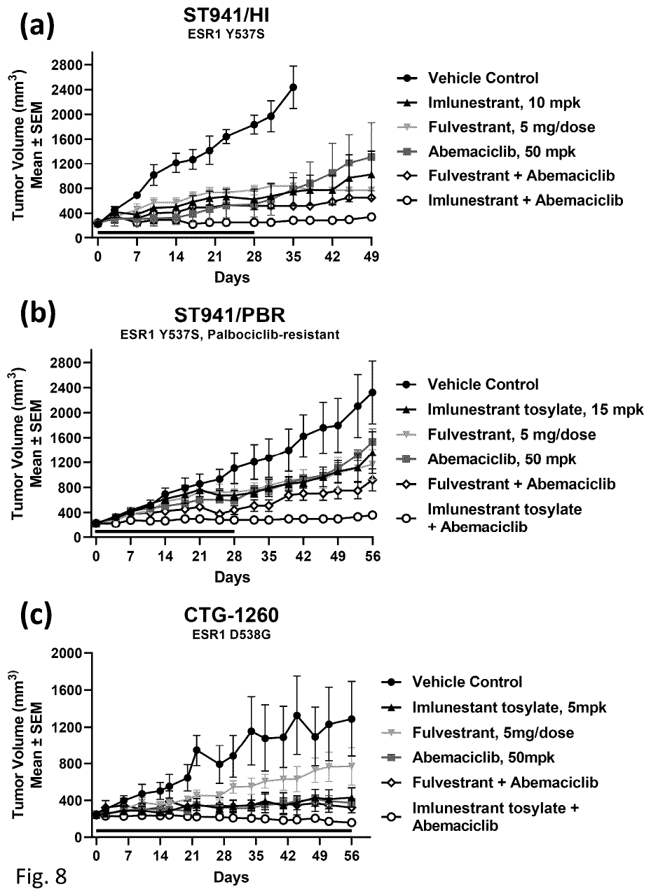

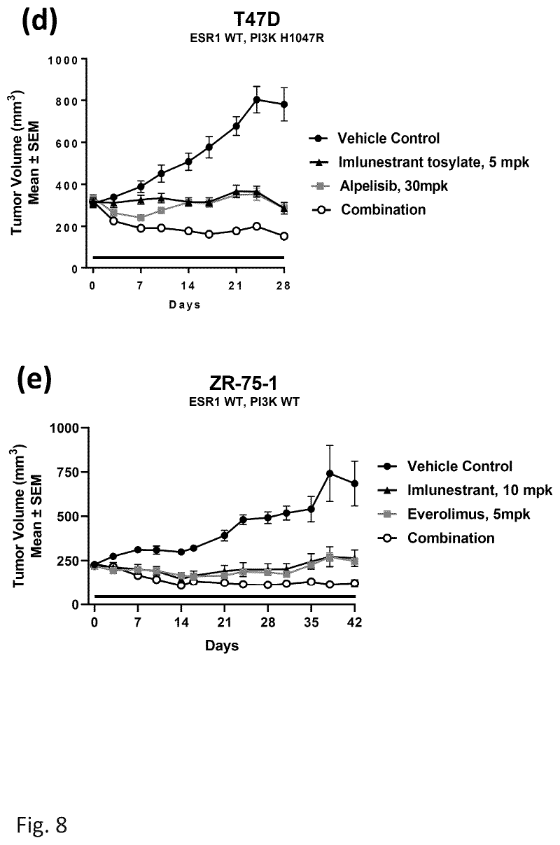

### Example 8. In Vivo Efficacy in a ST941/HI (ESR1 Y537S) Xenograft Model, a ST941/PBR (ESR1 Y537S, Palbociclib Resistant) Xenograft Model, a CTG-1260 (ESR1 D538G) Xenograft Model, a T47D (ESR1 WT, PI3K H1047R) Xenograft Model, and a ZR-75-1 (ESR1 WT, PI3K WT) Xenograft Model (FIG. 8)

For cell derived xenograft (CDX) models T47D and ZR-75-1, 5×10⁶ cells were resuspended in 1:1 HBSS+Matrigel and subcutaneously injected into the rear right flank of female NOD SCID mice (Envigo). For the T47D model, 170-estradiol pellets (Innovative Research) were implanted subcutaneously 24 hours prior to cell line injection. For the ZR-75-1 model, animals were administered 50 μl estradiol valerate (Delestrogen) intramuscular injection (10 mg/mL) and then once every 14 days for the duration of the study. Dosing was initiated when tumors reached sizes of 250 to 350 mm³ For patient derived xenograft (PDX) models, tumor fragments from ST941/HI and ST941/PBR (START) or CTG-2432, CTG-1260 and CTG-1211 (Champions Oncology) models were harvested from host animals and implanted into immune-deficient mice (Jackson Laboratories), and the studies were initiated when the tumor volumes reached 125 to 250 mm³ (ST941 models) or 150 to 300 mm³ (CTG models). At the start of treatment, animals were randomized into groups of five and dosed orally daily with imlunestrant, imlunestrant tosylate, abemaciclib mesylate, alpelisib, or everolimus alone or in combination as indicated in the figure. Fulvestrant was dosed once weekly by subcutaneous injection. Imlunestrant, imlunestrant tosylate, and abemaciclib mesylate were synthesized internally at Eli Lilly and Company. Clinical grade Fulvestrant (Faslodex) purchased from TEVA (NDC #0591-5019-2). Alpelisib and everolimus were obtained from external vendors and characterized for purity and identity. Imlunestrant and imlunestrant tosylate were solubilized in 1% HEC/0.25% Tween 80/0.05% Antifoam; abemaciclib was solubilized in 1% HEC in 25 mM Phosphate Buffer, pH 2; alpelisib was solubilized in 10% 2-Hydroxypropyl-β-Cyclodextrin in Sterile Water; and everolimus was solubilized in a 50 mg/ml stock solution in 200 proof ethanol just prior to dilution in 5% Dextrose in Sterile Water. Tumor growth inhibition was determined by tumor volume measurement performed twice per week during treatment. Body weight measurements were taken as a general measure of toxicity whenever tumor volume was measured.

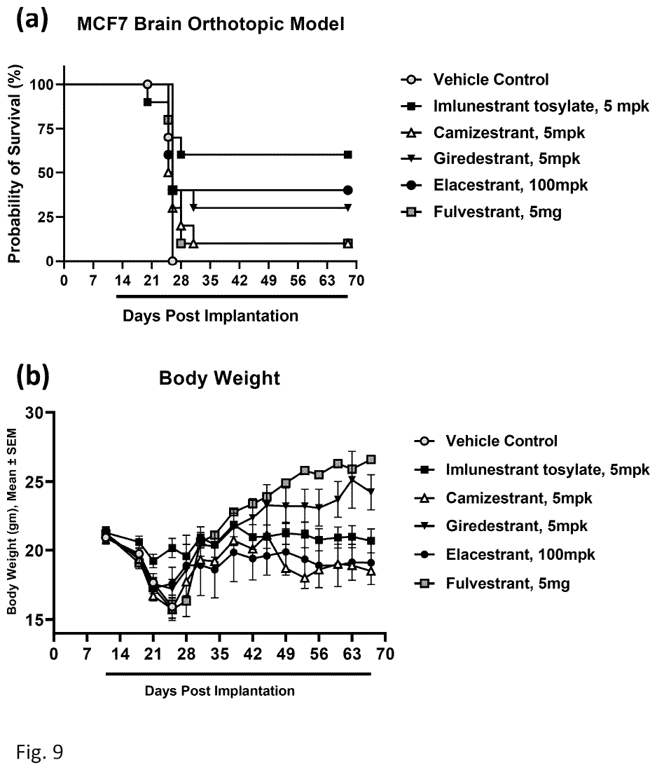

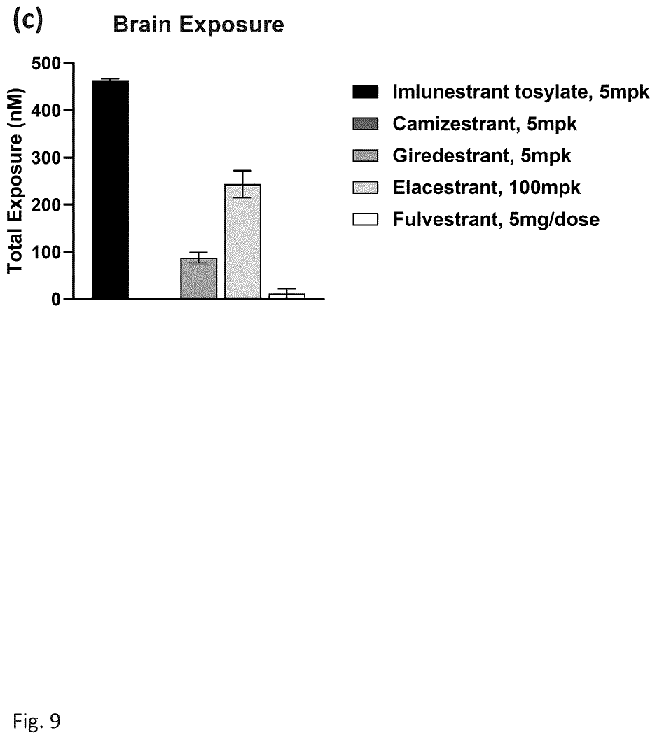

### Example 9. Probability of Survival, Body Weight, and Total Exposure in MCF Brain Orthotopic Model (FIG. 9)

Survival Analysis: MCF-7 cells stably expressing luciferase (MCF7-luc, internally generated) were implanted orthotopically into the brains of female NOD SCID mice (Envigo). 170-estradiol pellets were implanted subcutaneously twenty-four hours prior to cell implantation. Mice were randomized into groups of 10, and dosing was initiated 12 days after implantation. Imlunestrant tosylate was synthesized internally at Eli Lilly and Company. Elacestrant and fulvestrant were purchased externally, and giredestrant, and camizestrant were synthesized and characterized for purity at a CRO. Imlunestrant tosylate was solubilized in 1% HEC/0.25% Tween 80/0.05% Antifoam; camizestrant and giredestrant were solubilized in 20% Captisol in 25 mM Phosphate Buffer, pH 2 with 1 molar equivalent of 1N HCl; elacestrant was solubilized in 20% Captisol in 25 mM Phosphate Buffer, pH 2, and fulvestrant was solubilized in 10% DMSO in corn oil. All compounds were dosed orally once daily, except for fulvestrant which was dosed once weekly by subcutaneous injection. Body weight measurements were taken twice weekly as a general measure of toxicity.

Brain Exposure Analysis: Non-tumor bearing NOD SCID mice (Envigo, n=3 mice/group) were dosed with all compounds orally once daily for seven days, except for fulvestrant, which was dosed once by subcutaneous injection. The brains from all treatment groups were collected on day 8, snap frozen and sent to Q2 for exposure analysis.

### CONCLUSIONS

Imlunestrant is a potent degrader of ERα, inhibits cell growth in both ESR1 WT and mutant ER+BC cell lines, and suppresses ER-mediated pathways. Imlunestrant combined with other chemotherapeutic agents demonstrates additive or synergistic combination activity in multiple cell lines. PK/PD analysis of imlunestrant in vivo shows dose dependent exposure in multiple tissues, persistent exposure over time, sustained PGR gene expression inhibition, and reduction of ERα and PGR by immunohistochemistry. Imlunestrant is efficacious in ESR1 WT and mutant CDX and PDX models and exhibits enhanced efficacy in both WT and mutant PDX models when combined with other chemotherapeutic agents. Imlunestrant shows sustained exposure in the brain, demonstrating its ability to effectively cross the blood-brain barrier. In an ER+ brain orthotopic mouse model, imlunestrant treatment prolonged overall survival compared to control, fulvestrant and alternative SERD therapies.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1.-49. (canceled)

50. A method of inhibiting metastatic progression of cancer in the brain of a subject who has an ER+ breast cancer, the method comprising administering to the subject an effective amount of imlunestrant or a pharmaceutically acceptable salt thereof.

51. The method of claim 50, wherein the effective amount of imlunestrant or a pharmaceutically acceptable salt thereof further inhibits growth and/or survival of metastatic cancer cells in the brain of the subject.

52. The method of claim 50, wherein the effective amount of imlunestrant or a pharmaceutically acceptable salt thereof further prevents brain metastases in the subject.

53. The method of claim 50, wherein the effective amount of imlunestrant or a pharmaceutically acceptable salt thereof further reduces the risk of occurrence or development of brain metastases in the subject.

54. The method of claim 50, wherein the effective amount of imlunestrant or a pharmaceutically acceptable salt thereof further reduces the risk of developing detectable brain metastases in the subject.

55. The method of claim 50, wherein the effective amount of imlunestrant or a pharmaceutically acceptable salt thereof further delays the onset of brain metastases in the subject.

56. The method of claim 50, wherein the cancer is ER+/HER2+ breast cancer.

57. The method of claim 50, wherein the cancer is ER+/HER2− breast cancer.

58. The method of claim 50, wherein the cancer expresses the wild-type ER protein.

59. The method of claim 50, wherein the cancer expresses a mutant ER protein.

60. The method of claim 59, wherein the mutant ER protein comprises one or more mutations selected from S47R, A65V, N69K, P147Q, G160D, P222S, R233G, K252N, R271K, E279V, S282C, A293V, K303R, S329Y, G344D, L370F, E380Q, V392I, M396V, N407I, V418E, G420D, M421V, S432L, M437I, G442R, F461V, S463P, L466Q, E471D, V478L, R503W, E523Q, H524L, K531E, N532K, V533M, V534E, P535H/T, L536H/Q/P/R, Y537C/D/H/N/S, D538G/N, L540Q, E542G, A546D/T, L549P, R555C/H, G557R, T570I, S576L, A593D, and combinations thereof.

61. The method of claim 50, wherein the subject has metastatic breast cancer.

62. The method of claim 50, wherein the subject has brain metastatic breast cancer.

63. The method of claim 50, wherein the subject was previously treated for prior metastases.

64. The method of claim 50, wherein the subject has a primary cancer associated with an increased risk and/or an increased likelihood of metastatic progression.

65. The method of claim 50, wherein the subject was not known to have brain metastases prior to administering the imlunestrant or a pharmaceutically acceptable salt thereof.

66. The method of claim 50, wherein the subject has brain metastasized breast cancer (BMBC) and the effective amount of imlunestrant or a pharmaceutically acceptable salt thereof treats symptoms of BMBC selected from headaches, memory loss, changes in mood, changes in behavior, changes in personality, impaired judgment, dizziness, balance problems, weakness or paralysis in one side of the body (hemiparesis), numbness or tingling within the extremities, blurred vision, double vision, loss of vision, seeing flashes of light, slurred speech, nausea, vomiting, strokes, and seizures.

67. The method of claim 50, further comprising administering to the subject an additional anti-cancer agent selected from a CDK 4/6 inhibitor, a PI3K inhibitor, or a combination thereof.

68. The method of claim 67, wherein the CDK 4/6 inhibitor is abemaciclib.

69. The method of claim 68, wherein the subject is CDK 4/6 inhibitor naïve.
